SEQUENCE LISTING (11 sequences)

SEQ ID NO: 1 - DNA, 1984 bp, Homo sapiens
cccgagagcgcccattcacccccctcacccacctccccgcgttcccacttccccgcactctgacccggccggacgcccctcccctatcttgccgcccgccccctccagggggctctgctcccaccccagggagcccatccgacctctgcttgacttcccgccgcttccttcaggggcctcggctcatcgggtgcccctccccaaacttccaacccgtttgctccaggagttcctgccccatccccgagggcgcccaaatagccacactgtgtcctcctgtagtcgccgccccctgacctagcgcgacccagcgcccccgcccatgtccccccactcacctcccccggggggcgtggtgagtcgcggtctgttctcacggacggtccccgtccagcctgcgcttcgccggggccctcatctgctttcccgccaccctatcactcccttgccgtccaccctcggtcctcatggtcccagagccgggcccaaccgccaatagcaccccggcctggggggcagggccgccgtcggccccggggggcagcggctgggtggcggccgcgctgtgcgtggtcatcgcgctgacggcggcggccaactcgctgctgatcgcgctcatctgcactcagcccgcgctgcgcaacacgtccaacttcttcctggtgtcgctcttcacgtctgacctgatggtggggctggtggtgatgccgccggccatgctgaacgcgctgtacgggcgctgggtgctggcgcgcggcctctgcctgctctggaccgccttcgacgtgatgtgctgcagcgcctccatcctcaacctctgcctcatcagcctggaccgctacctgctcatcctctcgccgctgcgctacaagctgcgcatgacgcccctgcgtgccctggccctagtcctgggcgcctggagcctcgccgctctcgcctccttcctgcccctgctgctgggctggcacgagctgggccacgcacggccacccgtccctggccagtgccgcctgctggccagcctgccttttgtccttgtggcgtcgggcctcaccttcttcctgccctcgggtgccatatgcttcacctactgcaggatcctgctagctgcccgcaagcaggccgtgcaggtggcctccctcaccaccggcatggccagtcaggcctcggagacgctgcaggtgcccaggaccccacgcccaggggtggagtctgctgacagcaggcgtctagccacgaagcacagcaggaaggccctgaaggccagcctgacgctgggcatcctgctgggcatgttctttgtgacctggttgcccttctttgtggccaacatagtccaggccgtgtgcgactgcatctccccaggcctcttcgatgtcctcacatggctgggttactgtaacagcaccatgaaccccatcatctacccactcttcatgcgggacttcaagcgggcgctgggcaggttcctgccatgtccacgctgtccccgggagcgccaggccagcctggcctcgccatcactgcgcacctctcacagcggcccccggcccggccttagcctacagcaggtgctgccgctgcccctgccgccggactcagattcggactcagacgcaggctcaggcggctcctcgggcctgcggctcacggcccagctgctgcttcctggcgaggccacccaggaccccccgctgcccaccagggccgctgccgccgtcaatttcttcaacatcgaccccgcggagcccgagctgcggccgcatccacttggcatccccacgaactgacccgggcttggggctggccaatggggagctggattgagcagaacccagaccctgagtccttgggccagctcttggctaagaccaggaggctgcaagtctcctagaagccctctgagctccagaggggtgcgcagagctgaccccctgctgccatctccaggccccttacctgcagggatcatagctgactcaga

SEQ ID NO: 2 - protein, 440 aa, Homo sapiens
MVPEPGPTANSTPAWGAGPPSAPGGSGWVAAALCVVIALTAAANSLLIALICTQPALRNTSNFFLVSLFTSDLMVGLVVMPPAMLNALYGRWVLARGLCLLWTAFDVMCCSASILNLCLISLDRYLLILSPLRYKLRMTPLRALALVLGAWSLAALASFLPLLLGWHELGHARPPVPGQCRLLASLPFVLVASGLTFFLPSGAICFTYCRILLAARKQAVQVASLTTGMASQASETLQVPRTPRPGVESADSRRLATKHSRKALKASLTLGILLGMFFVTWLPFFVANIVQAVCDCISPGLFDVLTWLGYCNSTMNPIIYPLFMRDFKRALGRFLPCPRCPRERQASLASPSLRTSHSGPRPGLSLQQVLPLPLPPDSDSDSDAGSGGSSGLRLTAQLLLPGEATQDPPLPTRAAAAVNFFNIDPAEPELRPHPLGIPTN

SEQ ID NO: 3 - DNA, 24 bp, Artificial Sequence
  Synthetic primer
tgctagctcgcggcctctgtctgc

SEQ ID NO: 4 - DNA, 22 bp, Artificial Sequence
  Synthetic primer
catccctgggcgtggtgtcctg

SEQ ID NO: 5 - DNA, 20 bp, Artificial Sequence
  Synthetic primer
gccgccaattcgctgctgat

SEQ ID NO: 6 - DNA, 23 bp, Artificial Sequence
  Synthetic primer
acgccggacgccacgaggacata

SEQ ID NO: 7 - protein, 6 aa, Artificial Sequence
  Synthetic peptide
TLESIM

SEQ ID NO: 8 - protein, 5 aa, Artificial Sequence
  Synthetic peptide
EYNLV

SEQ ID NO: 9 - protein, 5 aa, Artificial Sequence
  Synthetic peptide
DCGLF

SEQ ID NO: 10 - DNA, 36 bp, Artificial Sequence
  Synthetic primer
gatcaagcttccatggcgtgctgcctgagcgaggag

SEQ ID NO: 11 - DNA, 53 bp, Artificial Sequence
  Synthetic primer
gatcggatccttagaacaggccgcagtccttcaggttcagctgcaggatggtg

(19) United States
(12) Patent Application Publication
(10) Pub. No.: US 2011/0319451 A1
(43) Pub. Date: Dec. 29, 2011

(54) 5-Ht6 Receptor and Modulators Thereof for the Treatment of Insulin-Related Disorders

(76) Inventors: James N. Leonard, San Diego, CA (US); Zhi-Liang Chu, San Diego, CA (US); Brian M. Smith, San Diego, CA (US)

(21) Appl. No.: 13/055,128

(22) PCT Filed: Jul. 22, 2009
(86) PCT No.: PCT/US2009/004247

§ 371 Date: Sep. 9, 2011

Related U.S. Application Data
(60) Provisional application No. 61/135,667, filed on Jul. 23, 2008.

(51) Int. Cl. A61K 31/4439; A61P 3/10; C12Q 1/02; A61P 3/04; A61P 3/08; A61P 5/48; A61P 3/06

(52) U.S. Cl. 514/339; 435/29

(57) ABSTRACT

The present invention relates to a method for identifying a glycemic stabilizing compound, by: a) contacting a candidate compound with a 5-HT₆ receptor, b) determining whether 5-HT₆ receptor functionality is increased, and c) determining whether a glycemic marker is directly modulated, where an increase in 5-HT₆ receptor functionality and direct modulation of a glycemic marker identifies the candidate compound as the glycemic stabilizing compound. In addition, the invention relates to a method for identifying a glycemic stabilizing compound, by: a) contacting a single dose of a candidate compound with a 5-HT₆ receptor, b) determining whether 5-HT₆ receptor functionality is increased, and c) determining whether a glycemic marker is directly modulated, where an increase in 5-HT₆ receptor functionality and direct modulation of a glycemic marker identifies the candidate compound as the glycemic stabilizing compound. Further, the invention relates to a method for identifying a glycemic stabilizing compound, by: a) contacting a candidate compound with a 5-HT₆ receptor in an isolated cell, b) determining whether 5-HT₆ receptor functionality is increased, and c) determining whether a glycemic marker is directly modulated, where an increase in 5-HT₆ receptor functionality and direct modulation of a glycemic marker identifies the candidate compound as the glycemic stabilizing compound.

### FIELD OF THE INVENTION

The present invention relates to methods for identifying a glycemic stabilizing compound, for example, a compound that controls insulin secretion or blood glucose levels, by determining whether a compound modulates 5-HT₆ receptor functionality. Accordingly, compounds of the present invention are useful in the prophylaxis or treatment of insulin-related disorders such as, for example, impaired glucose tolerance or diabetes.

### BACKGROUND OF THE INVENTION

Cells use glucose as a main source of energy. Therefore, food is first broken down by the body to glucose prior to being utilized. Glucose is then released from the gut into the blood resulting in a rise in blood glucose levels. In response to this rise in glucose level, pancreatic β-islet cells increase their production and secretion of insulin. Insulin circulates through the blood and acts as a messenger, sending a signal to insulin responsive organs such as the adipose tissue, muscle and liver, to increase their intake of glucose. In this way a rise in blood glucose is accompanied by a subsequent increase in insulin secretion from β-cells. It is the rise in insulin that acts to return blood glucose levels to normal. In healthy individuals blood glucose levels are kept fairly constant. This state of equilibrium, called normoglycemia (normal glucose level) is tightly controlled by insulin.

In diseases such as diabetes this tight regulation of blood glucose level is lost, leading to the increased blood glucose levels observed in diabetics. A state of hyperglycemia (high glucose level) can occur due to an insufficient production of insulin by the pancreatic β-cells and/or through inadequate uptake of glucose by target organs such as muscle, liver and fat. The end result is an increase in blood glucose level. Thus, diabetes can be thought of as the result of two types of impairment: impaired insulin secretion from the β-cells and impaired insulin sensitivity by the major insulin responsive organs. This impaired insulin sensitivity, also known as insulin resistance (because the organs are resistant to the effects of insulin), means that more insulin is required in order for the target organs to increase their glucose uptake. Insulin resistance leads to increased pressure on the β-cells because the β-cells need to increase their insulin secretion to compensate for insulin resistance. This is an escalating problem leading first to impaired glucose tolerance and; eventually, complete loss of insulin secretion due to the inability of the pancreas to keep up with the ever-increasing demand for insulin.

Diabetes is a diagnostic term for a group of disorders characterized by abnormal glucose homeostasis resulting in elevated blood glucose. There are many types of diabetes, but the two most common are type 1, also referred to as insulin-dependent diabetes mellitus or IDDM, and type 2, also referred to as non-insulin-dependent diabetes mellitus or NIDDM. Type 1 diabetes is mainly a disease with a young age of onset, and is due to the destruction of the insulin secreting β-cells in the pancreas by the immune system. In this case the body fails to recognize the pancreatic β-cells as being self and destroys its own cells. With the destruction of the β-cells there is a complete loss of insulin secretion and so affected individuals have an absolute dependency on insulin for survival. Type 2 diabetes is mainly a disease with a later age of onset, usually after the age of 40, but in recent years it is more common to find younger people being diagnosed with type 2 diabetes. It is mainly characterized by insulin resistance and beta cell exhaustion and is often associated with obesity. Type 2 diabetes is more common than type 1 diabetes and accounts for 90-95% of all diabetes cases diagnosed worldwide.

Inappropriate control of blood glucose level is also a characteristic of diseases other than diabetes such as Syndrome X (also called metabolic syndrome) and obesity. For example, one of the characteristics of Syndrome X is insulin resistance or glucose intolerance. In addition, obesity is characterized by hyperinsulinemia and insulin resistance, a feature shared with type 2 diabetes. Further, obesity is a major risk factor for type 2 diabetes. The risk of developing type 2 diabetes is tripled in subjects 30% or more overweight, and three-quarters of type 2 diabetes patients are overweight.

Obesity, which is the result of an imbalance between caloric intake and energy expenditure, is highly correlated with insulin resistance and diabetes in experimental animals and humans. During early development of obesity, increased insulin secretion balances insulin resistance and protects patients from hyperglycemia (Le Stunff, et al., Diabetes 43:696-702 (1989)). However, over time, β cell function deteriorates and non-insulin-dependent diabetes develops in about 20% of obese individuals (Pederson, P., Diab. Metab. Rev. 5:505-509 (1989), and Brancati, F. L., et al., Arch. Intern. Med. 159:957-963 (1999)). Given its high prevalence in modern societies, obesity has thus become the leading risk factor for NIDDM (Hill, J. O., et al., Science 280:1371-1374 (1998)). However, the factors which predispose some patients to alteration of insulin secretion in response to fat accumulation are still under investigation. Unfortunately, effective long-term therapies to treat obesity are still not available.

A systematic review of the long-term effects of weight loss on diabetes outcomes in obese people, or for those at risk of developing type 2 diabetes, based on studies published between 1996 and 2001, showed that those with diabetes who lost weight intentionally significantly reduced their mortality risks by 25% (Aucott, L. Poobalan, A., Smith, W. C. S., Avenell, A., Jung, R., Broom, J., Grant, A. M. Diabetes, Obesity & Metabolism 6:85-94 (2004)). Additionally, weight loss of 9-13 kg was most protective. Patients with the risk of developing diabetes due to either family history of diabetes or impaired glucose tolerance, saw a reduction in this risk. Those with large weight losses achievable with surgical interventions reduced their risk by at least 63%. Metabolic handling of glucose improved in 80% of those already with type 2 diabetes who lost weight. In the Nurses Health study it was found that as little as a 4.2 kg loss in body weight sustained for an average of 3.2 years could reduce the progression to type 2 diabetes by 50% (Tuomilehto J, Lindstrom J, Eriksson J G, Walle T T, Hamalainen H, Ilanne-Parikka P, Keinanen-Kivaanniemi S, Laasko M, Louheranta A, Rastas M, Salminen V, and Uusitupa M. N Engl J Med 344:1343-1450, (2001)).

Diabetes afflicts several million people worldwide. In the United States alone, there are more than 20 million diabetics, with more than 600,000 new cases diagnosed each year. People with diabetes are at higher risk for heart disease, blindness, kidney failure, infection, extremity amputations, and other conditions. It is estimated that the direct medical expenditures and indirect expenditures attributable to diabetes in the United States were $132 billion in 2002. Taken together, diabetes complications are one of the nation's leading causes of death.

Therapies do exist to treat diabetes, such as α-glucosidase inhibitors, biguanides, thiazolidinediones, meglitinides, sulfonylureas, incretin-based therapies and exogenous insulin. However, these therapies have limited effectiveness and are associated with significant safety and tolerability issues such as risk for hypoglycemic episodes, weight gain, gastrointestinal disturbances and anemia. In addition, many of the treatment options require injection or multiple daily dosings which present compliance challenges.

In addition to disorders that benefit from increasing insulin secretion such as diabetes, there are a number of disorders that can benefit from decreasing insulin secretion. For example, a decrease in insulin secretion can result in an increase in blood glucose which is needed during hypoglycemia. In addition, for example, decreasing insulin secretion can be useful for a patient with an insulinoma, which is a tumor that secretes excess insulin. Insulin can also serve as a growth factor for certain tumors. Further, caloric restriction is known to down-regulate insulin secretion and this may be a mediator of caloric restriction's favorable impact on longevity. Thus, a reduction in insulin secretion can be beneficial to treat aging. In all these cases, a reduction in insulin levels can be beneficial.

Thus, there exists a need for the identification of an agent which safely and effectively modulates insulin secretion and/or blood glucose levels for the treatment of insulin-related disorders such as hypoglycemia, an insulin-secreting or insulin-dependent tumor, aging, syndrome X, insulin resistance, impaired glucose tolerance, or diabetes. The present invention satisfies this need and provides related advantages as well.

### SUMMARY OF THE INVENTION

Applicants disclose herein that the 5-HT₆ receptor is expressed in transformed mouse L cells (endocrine cells) as well as brain, duodenum, small intestine and colon. In addition, Applicants disclose that a 5-HT₆ receptor agonist directly improves oral glucose tolerance and enhances insulin release in mice. Further Applicants disclose robust insulin release in mice treated with a combination of a Dipeptidyl Peptidase IV (DPP-IV) inhibitor and a 5-HT₆ receptor agonist. In addition, Applicants disclose that a 5-HT₆ receptor agonist improves oral glucose tolerance in diabetic db/db mutant mice.

In a first aspect, the invention features a method for identifying a glycemic stabilizing compound, comprising: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound. In one embodiment, the modulation of the glycemic marker is first detected within less than 24 hours of the contacting of the candidate compound with the 5-HT₆ receptor. In another embodiment, said 5-HT₆ receptor is human. In a further embodiment, said determining comprises a second messenger assay. In a yet further embodiment, said glycemic stabilizing compound decreases blood glucose concentration. In another embodiment, said glycemic stabilizing compound increases insulin secretion. In one embodiment, said glycemic marker is glucose. In another embodiment, said glycemic marker is insulin.

In a second aspect, the invention features a method for identifying a glycemic stabilizing compound, comprising: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound. In one embodiment, said 5-HT₆ receptor is human. In another embodiment, said determining comprises a second messenger assay. In a further embodiment, said glycemic stabilizing compound decreases blood glucose concentration. In a yet further embodiment, said glycemic stabilizing compound increases insulin secretion. In one embodiment, said glycemic marker is glucose. In another embodiment, said glycemic marker is insulin.

In a third aspect, the invention features a method for identifying a glycemic stabilizing compound, comprising: a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound. In one embodiment, said 5-HT₆ receptor is human. In another embodiment, said determining comprises a second messenger assay. In a further embodiment, said glycemic stabilizing compound decreases blood glucose concentration. In a yet further embodiment, said glycemic stabilizing compound increases insulin secretion. In one embodiment, said glycemic marker is glucose. In another embodiment, said glycemic marker is insulin.

In a fourth aspect, the invention features a method for treating or preventing an insulin-related disorder in an individual in need thereof, comprising administering to said individual an effective amount of the glycemic stabilizing compound of the first, second or third aspect. In one embodiment, said insulin-related disorder is insulin resistance, impaired glucose tolerance, or diabetes. In another embodiment, said individual is a human. In a further embodiment, a method of the fourth aspect further comprises administering to said individual an effective amount of an agent used for the treatment of diabetes, blood lipid disorders, or obesity in combination with an effective amount of the glycemic stabilizing compound of the first, second or third aspect.

In a fifth aspect, the invention features a method for decreasing blood glucose levels in an individual in need thereof, comprising administering to the individual an effective amount of the glycemic stabilizing compound of the first, second or third aspect.

In a sixth aspect, the invention features a method for increasing insulin secretion in an individual in need thereof, comprising administering to the individual an effective amount of the glycemic stabilizing compound of the first, second or third aspect. In one embodiment, increasing insulin secretion in an individual is in a glucose dependent manner.

### BRIEF DESCRIPTION OF THE DRAWINGS

FIG. 1 shows RT-PCR analysis of mouse 5-HT₆ receptor expression in mouse tissues and transformed endocrine cells.

FIG. 2 shows RT-PCR analysis of mouse 5-HT₆ receptor expression in various tissues from three different ob/ob mutant mice.

FIG. 3 shows RT-PCR analysis of mouse 5-HT₆ receptor expression in mouse pancreas from ob/ob mutant mice and C57Bl/6 wild-type mice. Mouse brain tissue and mouse genomic DNA are used as positive controls for 5-HT₆ receptor expression.

FIG. 4 shows the result of cyclase assays for a 5-HT₆ receptor agonist.

FIG. 5 shows that a 5-HT₆ receptor agonist inhibits glucose excursion in an oral glucose tolerance test (oGTT) in wild-type mice (upper panel) and increases insulin release in 5-HT₆ receptor agonist treated mice (lower panel).

FIG. 6 shows robust insulin release in mice treated with a combination of a DPP-IV inhibitor and a 5-HT₆ receptor agonist.

FIG. 7 shows that a 5-HT₆ receptor agonist does not cause hypoglycemia in C57/Bl/6 mice.

FIG. 8 shows inhibited glucose excursion in an oGTT in diabetic db/db mice treated with a 5-HT₆ receptor agonist.

### DETAILED DESCRIPTION

Although a number of receptor classes exist in humans, the most abundant and currently therapeutically relevant is represented by the G protein-coupled receptor (GPCR) class. It is estimated that there are some 30,000-40,000 genes within the human genome, and of these, approximately 2% are estimated to code for GPCRs. GPCRs represent an important area for the development of pharmaceutical products: from approximately 20 of the 100 known GPCRs, approximately 60% of all prescription pharmaceuticals have been developed.

GPCRs share a common structural motif, having seven sequences of between 22 to 24 hydrophobic amino acids that form seven alpha helices, each of which spans the membrane (each span is identified by number, i.e., transmembrane-1 (TM-1), transmembrane-2 (TM-2), etc.). The transmembrane helices are joined by strands of amino acids between transmembrane-2 and transmembrane-3, transmembrane-4 and transmembrane-5, and transmembrane-6 and transmembrane-7 on the exterior, or “extracellular” side, of the cell membrane (these are referred to as “extracellular” regions 1, 2 and 3 (EC-1, EC-2 and EC-3), respectively). The transmembrane helices are also joined by strands of amino acids between transmembrane-1 and transmembrane-2, transmembrane-3 and transmembrane-4, and transmembrane-5 and transmembrane-6 on the interior, or “intracellular” side, of the cell membrane (these are referred to as “intracellular” regions 1, 2 and 3 (IC-1, IC-2 and IC-3), respectively). The “carboxy” (“C”) terminus of the receptor lies in the intracellular space within the cell, and the “amino” (“N”) terminus of the receptor lies in the extracellular space outside of the cell.

Generally, when a ligand binds with the receptor (often referred to as “activation” of the receptor); there is a change in the conformation of the receptor that facilitates coupling between the intracellular region and an intracellular “G-protein.” It has been reported that GPCRs are “promiscuous” with respect to G proteins, i.e., that a GPCR can interact with more than one G protein. See, Kenakin, T., 43 Life Sciences 1095 (1988). Although other G proteins exist, currently, Gq, Gs, Gi, Gz and Go are G proteins that have been identified. Ligand-activated GPCR coupling with the G-protein initiates a signaling cascade process (referred to as “signal transduction”). Under normal conditions, signal transduction ultimately results in cellular activation or cellular inhibition. Although not wishing to be bound to theory, it is thought that the IC-3 loop as well as the carboxy terminus of the receptor interact with the G protein.

Under physiological conditions, GPCRs exist in the cell membrane in equilibrium between two different conformations: an “inactive” state and an “active” state. A receptor in an inactive state is unable to link to the intracellular signaling transduction pathway to initiate signal transduction leading to a biological response. Changing the receptor conformation to the active state allows linkage to the transduction pathway (via the G-protein) and produces a biological response.

A receptor can be stabilized in an active state by a ligand or a compound such as a drug. Recent discoveries, including but not exclusively limited to modifications to the amino acid sequence of the receptor, provide means other than ligands or drugs to promote and stabilize the receptor in the active state conformation. These means effectively stabilize the receptor in an active state by simulating the effect of a ligand binding to the receptor. Stabilization by such ligand-independent means is termed “constitutive receptor activation.”

### The 5-HT₆ Receptor:

Several papers characterizeing the 5-HT₆ receptor are available in the literature (see, for example, the review paper Heal, D. J et al., Pharmacology & Therapeutics. 117:207-231 (2008) and the references cited therein).

The 5-HT₆ G-protein coupled receptor was first cloned from rat striatal tissue using RT-PCR in the early 1990's. Consistent with the structure of this family of receptors, the 5-HT₆ receptor is a seven transmembrane-spanning protein of about 440 amino acids. It was reported that the 5-HT₆ receptor was unusual for a member of the serotonin receptor family because its distribution was almost exclusively within the central nervous system. The mouse amino acid sequence shows 97% homology with the rat 5-HT₆ amino acid sequence and 89% similarity to the human sequence. Characterisation of the receptor in transfected cell lines demonstrated that it was positively coupled to adenylyl cyclase. Interestingly, using site-directed mutagenesis of the 5-HT₆ receptor, a constitutively active receptor was described, that demonstrated clear inverse agonism with some of the previously reported antagonists.

In the rat brain, the highest levels of 5-HT₆ receptor mRNA are present in the striatum (particularly nucleus accumbens), olfactory tubercle, hippocampus, cortex, cerebellum, hypothalamus and the amygdala. The highest density of mRNA within the hippocampus was found to be in the dentate gyrus, CA1, CA2 and CA3 regions. The 5-HT₆ receptor protein was found in the olfactory tubercle, cortex, striatum (particularly nucleus accumbens), hippocampus, cerebellum, thalamus, substantia nigra, superficial layer of the superior colliculus, motor trigeminal nucleus and facial nucleus and in the hypothalamus, which is the brain region responsible for the regulation of food intake and energy expenditure, and is an important site for the action of centrally-acting anti-obesity drugs.

The human 5-HT₆ receptor was cloned by Kohen and colleagues in 1996 and they found the highest expression of mRNA to be in the caudate nucleus, followed by the hippocampus and amygdala. Low expression levels were found in the thalamus, subthalamic nuclei and substantia nigra.

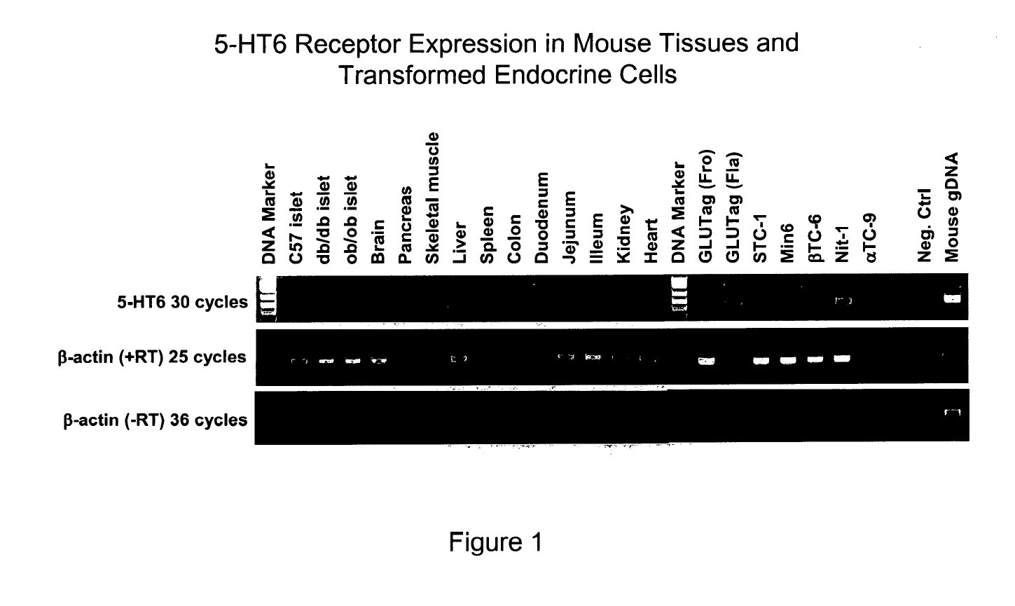

Applicants disclose herein that the 5-HT₆ receptor is expressed in mouse tissues and transformed endocrine cells (see FIGS. 1, 2 and 3). In addition to showing expression in the brain, Applicants show expression of the 5-HT₆ receptor in peripheral cells and tissues in the mouse. As disclosed herein, Applicants find expression of the 5-HT₆ receptor in a transformed mouse enteroendorine cell line designated GLUTag (see FIG. 1—GLUTag (Fro) and GLUTag (Fla) are two sublines obtained from Dr. Daniel Drucker at the University of Toronto). GLUTag cells are transformed L cells, which are specialized gut endocrine cells. The majority of L cells are classically thought to be located in the distal gut, predominantly the ileum and colon. The function of these cells is to synthesize and secrete gut hormones. There appears to be distinct populations of L cells that express glucagon-like peptide 1 (GLP-1) alone or co-express GLP-1 and peptide YY (PYY) or GLP-1 and cholecystokinin (CCK). GLP-1 is an incretin hormone which stimulates the release of insulin from pancreatic beta cells. GLUTag cells respond to the same secretagogues that normally regulate rat enteroendocrine cell function and appear highly differentiated when compared to the behavior of normal rat intestinal endocrine cells.

Interestingly, L cells in the gut have been shown to be proximal to enterochromaffin (EC) cells which are cells that are known to release serotonin. The release of serotonin from EC cells can provide a local source of ligand for the 5-HT₆ receptor expressed on L cells. This can allow for a paracrine type of regulation system to exist in the gut for the 5-HT₆ receptor.

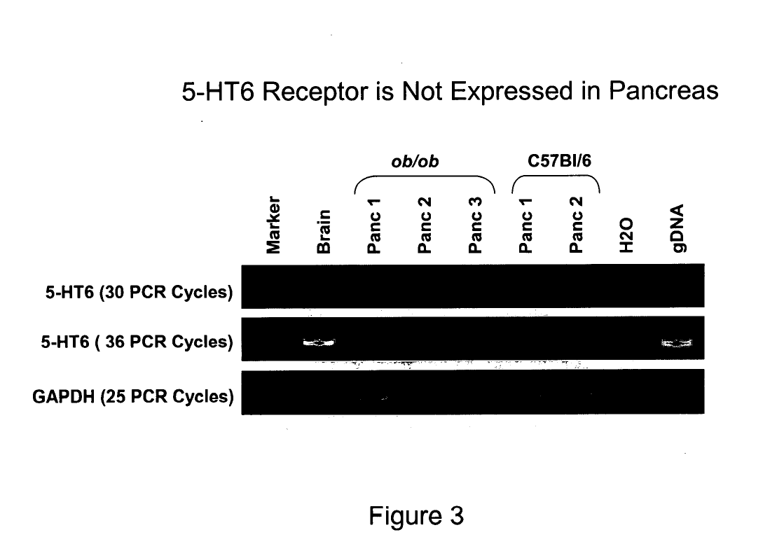

Applicants also show expression of the 5-HT₆ receptor in transformed beta islet cell lines such as Min6 and Nit-1 (FIG. 1). Beta islet cells are present in the pancreas; however, Applicants do not see expression of the 5-HT₆ receptor in isolated mouse islet cells (FIG. 1) or in the pancreas as a whole (FIG. 3). Applicants believe that expression of the 5-HT₆ receptor in transformed beta islet cells may be the result of the de-differentiated nature of the transformed cell line. If the 5-HT₆ receptor was truly expressed in beta islet cells, one would expect to see expression by PCR in the isolated islet cells or pancreas.

The pancreas is divided into lobules by connective tissue septae. Lobules are composed largely of grape-like clusters of exocrine cells called acini, which secrete digestive enzymes. Embedded within the pancreatic exocrine tissue are Islets of Langerhans, the endocrine component of the pancreas. Islets make up about 1% of the pancreas. Islets contain several cell types and are richly vascularized. The islet cell types include alpha and beta cells as well as other cell types. It is the beta islet cells that secrete insulin. Further, Applicants show expression of 5-HT₆ receptor in transformed alpha TC1-9 cells which are predominantly an alpha cell type from the pancreas. Again, the alpha TC1-9 cells are transformed cell and so are de-differentiated and thus express a wider array of genes than would be expected in the fully differentiated cell type.

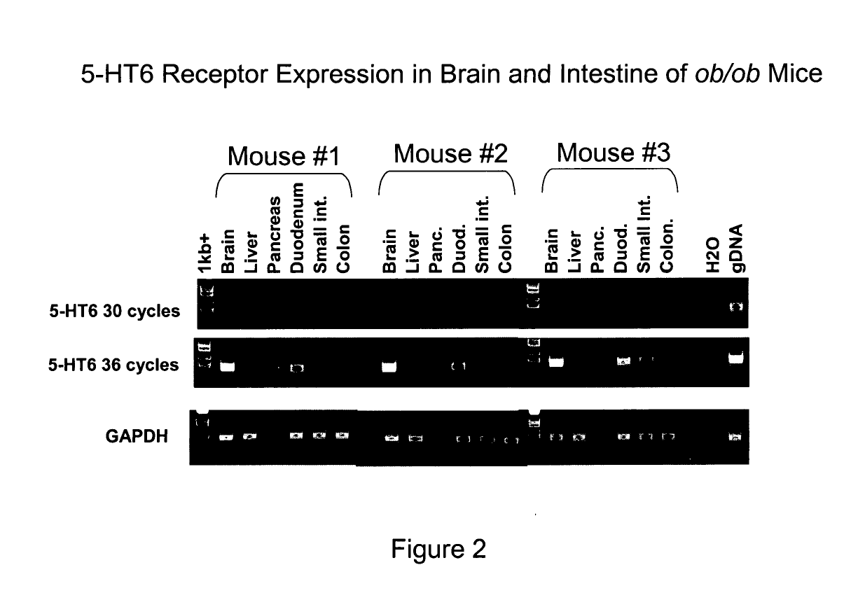

Applicants also disclose herein the presence of the 5-HT₆ receptor in the brain, duodenum, small intestine and colon of mutant obese (ob/ob) mice (FIG. 2). The presence of the 5-HT₆ receptor in the gut corroborates the expression Applicant's have shown in L cells (FIG. 1).

While much attention has been focused on the expression of the 5-HT₆ receptor in the brain, Applicants show expression of the 5-HT₆ receptor in peripheral tissues as well. This finding is notable because peripheral expression of the 5-HT₆ receptor must be taken into account when designing drugs that act on the receptor. For example, if one is targeting the 5-HT₆ receptor to treat a brain disease such as schizophrenia or Alzheimer's disease, care must be taken to look for side effects caused by interaction of the drug at a peripheral receptor. Conversely expression of the 5-HT₆ receptor in the brain must be considered when designing drugs that treat a peripheral disease such as diabetes. However, most drugs will not cross the blood brain barrier and so often one can target the 5-HT₆ receptor in the periphery without causing side effects from interaction with the receptor in the brain.

Soon after its discovery in 1993, the 5-HT₆ receptor became an attractive target for medicinal chemistry, since it was established that numerous tricyclic antipsychotics and antidepressants showed high affinity for that receptor. Agents that bind at human 5-HT₆ receptors with Ki values <50 nM include 5-methoxytryptamine, bromocriptine, octoclothepin, and the neuroleptics, clozapine, olanzapine, loxapine, chlorpromazine and fluphenazine. Many new 5-HT₆ ligands have been synthesized, amongst which, various selective agents have been identified.

The 5-HT₆ receptor has been shown to be involved in feeding behavior in rodent animal models. For example, injection of 5-HT₆ antisense oligonucleotides into the brain have been shown to result in decreased feeding behaviour in rats and decreased body weight. Moreover, it was reported that the small-molecule 5-HT₆ receptor antagonist, Ro 04-6790, at a high dose of 30 mg/kg i.p., significantly attenuated body weight-gain in growing rats when given daily for 3 days. In addition, the rate of weight regained after withdrawal from treatment was slow.

It has been reported that a 5-HT₆ receptor knock-out mouse was resistant to dietary-induced obesity when maintained on a high-fat diet. Effects on food intake and body weight in 5-HT₆ receptor knock-out mice have led to a view that 5-HT₆ receptor antagonists will evoke hypophagia and weight-loss only under conditions of high receptor occupancy. Preliminary pharmacology results for a high affinity small-molecule 5-HT₆ receptor antagonist, BVT 5182 (5-HT₆ Ki=0.2 nM), showed that when given acutely this compound dose-dependently reduced the food intake of ob/ob mice by enhancing satiety, and when given repeatedly, BVT 5182 produced a sustained reduction in food intake and weight-loss in DIO mice. The weight-loss evoked by BVT 5182 in DIO mice was shown to be accompanied by a reduction in visceral adiposity, and plasma leptin and insulin concentrations.

Several selective small-molecule 5-HT₆ ligands have been synthesized and tested in rodent models of obesity. PRX-07034 is a high affinity (5-HT₆ Ki=4 nM) 5-HT₆ receptor antagonist with 65 to >300-fold selectivity versus other 5-HT receptor subtypes, and with the exception of the dopamine D3 receptor (Ki=71 nM), it has ≧100-fold selectivity over 52 other G-protein-coupled receptors, ion channels and transporters. In acute studies, PRX-07034 inhibited food intake in normal lean and DIO female rats. Saccharin consumption experiments and a behavioural analysis of meal patterns revealed no aversive effect of PRX-07034 and a hypophagic mechanism that was consistent with an enhancement of satiety.

Since it is easy to suppress feeding in animals using drugs by a variety of mechanisms that are not clinically acceptable, e.g. compound-induced activation, sedation, stereotypy, nausea/malaise or taste aversion, it is important to rule out these mechanisms in the action of the 5-HT receptor modulators. It has been reported that BVT-5182 and PRX-07034 decrease food intake by enhancing satiety and not by inducing locomotor changes, taste aversion or nausea.

An interesting confounder in this field is that 5-HT₆ partial agonists do not increase food consumption and exacerbate obesity in rodent models; on the contrary, they cause hypophagia and reduce adiposity like 5-HT₆ receptor antagonists. For example, E-6837 is a partial agonist of the rat 5-HT₆ receptor and a full agonist of the cloned human 5-HT₆ subtype. Recently, Fisas et al. used DIO female rats to compare the effects of E-6837 on food consumption and body weight with those of the reference anti-obesity drug, sibutramine (Fisas et al., 2006 ibid). When cumulative weekly food intakes were calculated, E-6837 significantly decreased consumption during the first, second and third weeks of treatment, whereas sibutramine significantly reduced food intake only during the first week. This difference in the food reduction profiles of E-6837 and sibutramine is reflected in the rate and duration of active weight-loss on treatment. It was also shown that E-6837 caused gradual weight-loss in DIO rats that had not plateaued after 28 days of compound administration. In contrast, sibutramine produced most of its weight-loss during the first 2 weeks, and thereafter, the body weight curve of the sibutramine-treated rats paralleled that of the vehicle-treated controls. The weight-loss versus control after 28 days of administration was 11.0% for sibutramine and 15.7% for E-6837.

The finding that 5-HT₆ partial agonists and antagonists produce identical pharmacological effects in animal models is not restricted to their anti-obesity actions. An identical phenomenon has also been reported in the cognition field where 5-HT₆ receptor agonists as well as antagonists have been shown to improve performance of the novel object recognition task. This paradox is also not explained by artefacts generated by the use of different obesity models because PRX-07034 (a 5-HT₆ receptor antagonist) and E-6837 (a rat 5-HT₆ receptor partial agonist) have both been shown to reduce food intake and adiposity in the female DIO rat model. The technical difficulty of defining the absolute functionality of compounds, especially 5-HT₆ receptor ligands, with cloned receptors stably transfected in cell lines is known. Thus, it may be that the characterisation of some ligands as either partial agonists (or antagonists) may not hold true for native receptors in their physiological environment, and as a consequence, this apparent pharmacological anomaly may be no more than an artefact of the in vitro characterisation of these 5-HT₆ receptor ligands. On the other hand, it has been suggested that the 5-HT₆ receptor may rapidly down-regulate in response to agonist stimulation, or alternatively, serotonergic tone at these receptors may be high, leading to a predominantly antagonist action of 5-HT₆ partial agonists.

Just as obesity is a major causative factor in the development of insulin resistance, impaired glucose tolerance, Type 2 diabetes, hypertension, dyslipidaemia and a range of other metabolic disorders, moderate, intentional weight-loss has been shown to have a positive effect on morbidity and mortality. These findings relate to intentional weight-loss through diet, exercise and lifestyle modification and it is yet to be shown that anti-obesity drug therapy will have the same impact on cardio-metabolic status (i.e. visceral adiposity, increased glycaemic control, beneficial changes in plasma lipid profiles, reduced plasma uric acid concentrations and decreased blood pressure), and by inference, on morbidity and mortality. Obese rats and mice are not suitable models for studying all the above endpoints, particularly plasma lipoprotein fractions (HDL-cholesterol, LDL-cholesterol, and VLDL-cholesterol), but they are predictive indicators for improvements in several other cardio-metabolic risk factors. In addition to demonstrating that several of 5-HT₆ receptor antagonists and partial agonists decreased food intake and body weight in obese rodents, some investigations also incorporated measurements of cardio-metabolic risk factors. Although full body composition analyses have not been reported for all of these 5-HT₆ receptor ligands, it has been shown that weight-loss produced by E-6837 is due to a selective reduction in body fat with no changes in either water or protein content, indicating that this compound is not causing weight-loss through the clinically unacceptable routes of dehydration or cachexia (Fisas et al., 2006 ibid). Where fat pads have been weighed, decreases in visceral fat depots have been observed and this finding is of particular clinical relevance because visceral adiposity in man is a major driver of cardio-metabolic risk. Consistent with the observation that plasma leptin concentrations correlate with white adipose tissue mass, the reduction of adiposity in either DIO rats is accompanied by a significant fall in the plasma level of this hormone. Improvements in glycemic control were also observed with all of these 5-HT₆ ligands, whether via decreases in the plasma concentrations of insulin and glucose or via improved insulin sensitivity in an oral glucose tolerance test. The improvements in glycemic control were seen as a consequence of weight loss and were not contemplated as a direct action at the 5-HT₆ receptor.

Regarding potential side effects of 5-HT₆ receptor modulators, it is known that 5-HT₆ receptor antagonists improve several aspects of cognitive function, and consequently, these drugs could evoke cognitive side-effects when used in the treatment of obesity or other diseases. However, the actions of the 5-HT₆ receptor antagonists are pro-cognitive, and as such, their side-effects are predicted to be neutral and perhaps even beneficial. Furthermore, following the recent report that 5-HT₆ receptor agonists have pro-cognitive effects that are similar in magnitude to those of the 5-HT₆ receptor antagonists, if these compounds evoke cognitive side-effects they are also likely to be beneficial rather than deleterious. The only other potential CNS side-effects to have been revealed by preclinical research are related to depression and anxiety. However, the data appear to be contradictory.

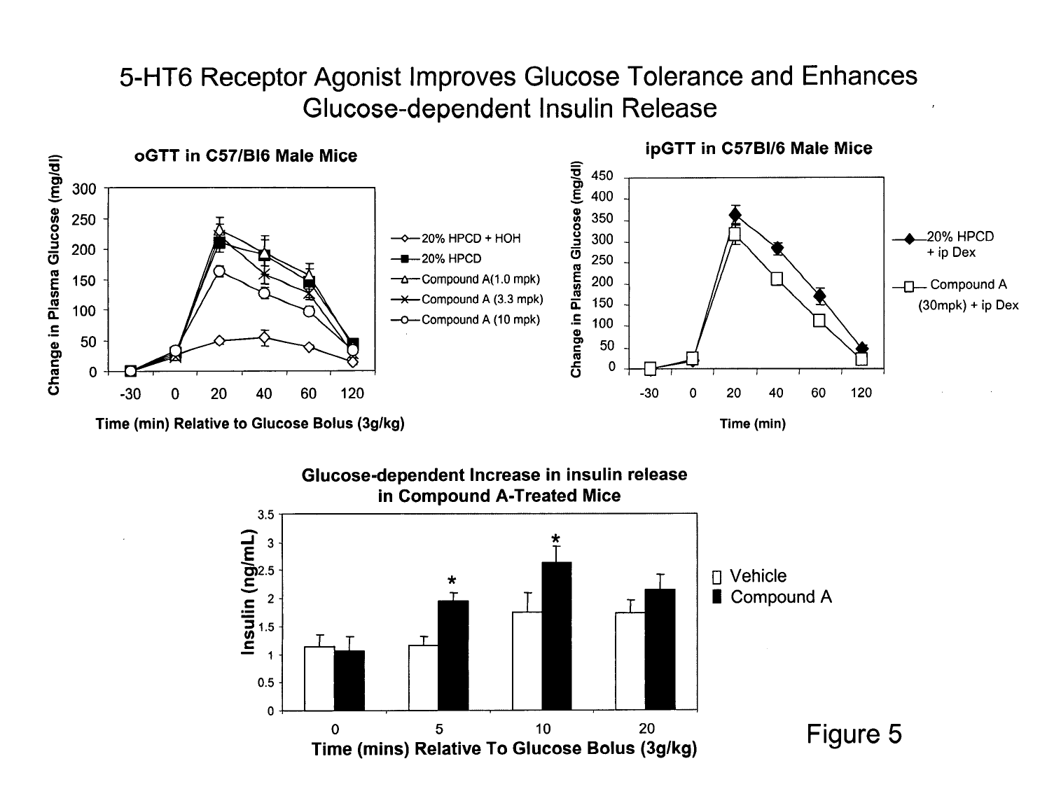

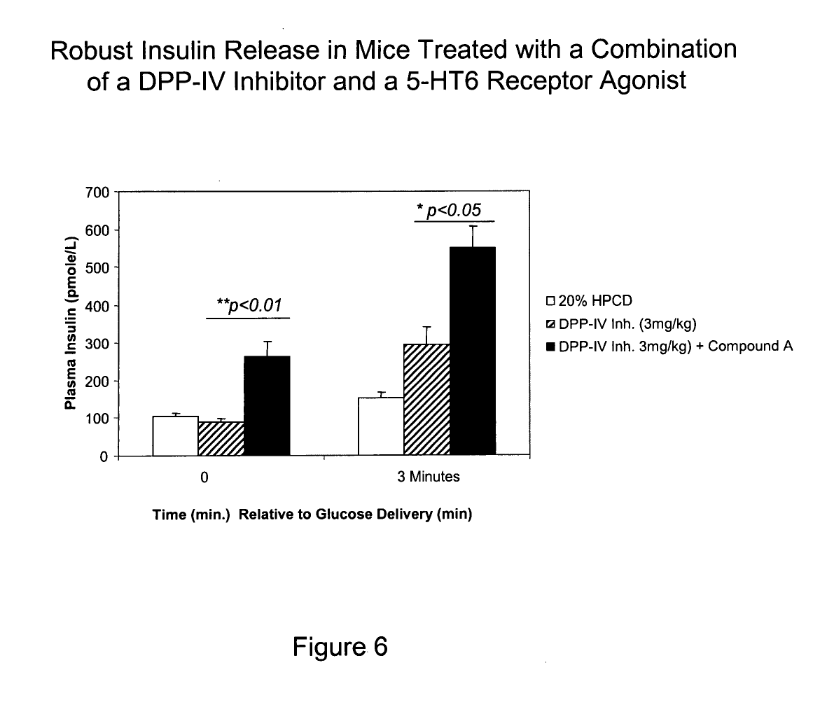

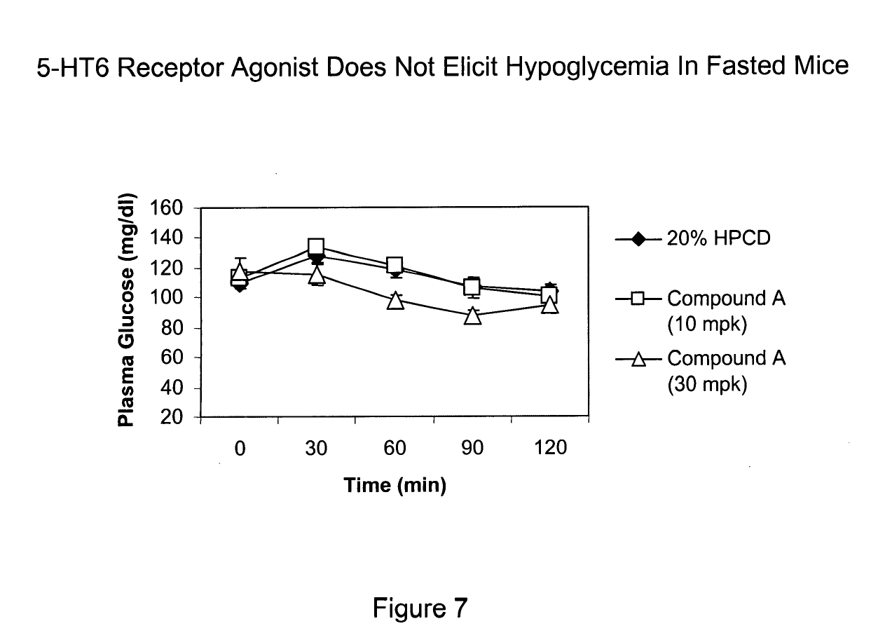

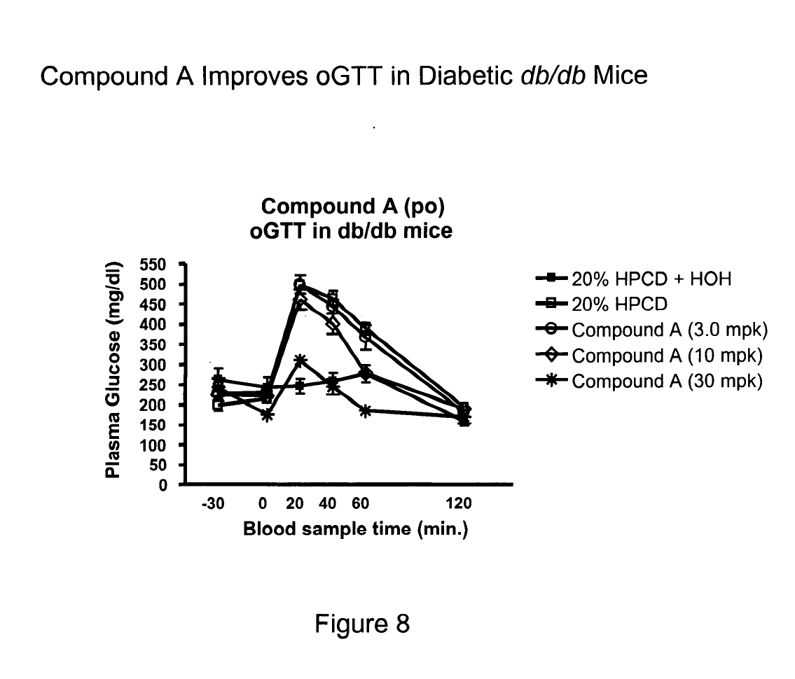

Applicants have disclosed herein that a 5-HT₆ receptor agonist inhibits glucose excursion in an oral glucose tolerance test (oGTT) in wild-type mice and increases insulin release in 5-HT₆ receptor agonist treated mice (FIG. 5). The increase in insulin release in 5-HT₆ receptor agonist treated mice is glucose dependent since there is no increase in insulin secretion at time 0, but there is an increase in insulin secretion after addition of the glucose bolus (i.e. time points after time 0—see FIG. 5, lower panel). In addition, Applicants disclose that mice treated with a combination of a DPP-IV inhibitor and a 5-HT₆ receptor agonist show robust insulin release (FIG. 6). In addition, Applicants show that a 5-HT₆ receptor agonist does not cause hypoglycemia in C57/Bl/6 mice (FIG. 7). Further Applicants disclose that treatment of db/db diabetic mice with a 5-HT₆ receptor agonist results in improved glucose tolerance in an oGTT (FIG. 8).

In contrast to the changes in oGTT seen by Fisas et al., (Fisas et al., ibid), Applicant's results were obtained using single dose treatments and the results were seen in a short time frame (less than 24 hours). Thus, Applicant's observe direct modulation of glycemic markers such as insulin and glucose. Weight loss is not the cause of the effects Applicants see on glucose and insulin levels in 5-HT₆ receptor agonist treated mice.

Applicant's discovery of a direct effect on glycemic markers through stimulation of the 5-HT₆ receptor now makes it viable to investigate 5-HT₆ receptor agonists and partial agonists as, for example, anti-diabetes drugs. Based on Applicants disclosure of a direct effect of 5-HT₆ receptor agonists on glucose and insulin levels, Applicants disclose use of the 5-HT₆ receptor as a method for screening for compounds that can directly regulate glucose and insulin levels in a mammal. Such compounds can be used in the treatment or prevention of an insulin-related disorder such as insulin resistance, impaired glucose tolerance or diabetes.

### DEFINITIONS

For clarity and consistency, the following definitions will be used throughout this patent document.

AGONIST refers to material, for example, a ligand or candidate compound, that activates a cellular response when it binds to the receptor. A cellular response can be, for example, enhancement of GTP binding to membranes or modulation of the level of a second messenger such as cAMP or IP3. In some embodiments, an AGONIST is material not previously known to activate the cellular response when it binds to the receptor (for example, to enhance GTPγS binding to membranes or to increase intracellular cAMP level). In some embodiments, an AGONIST is material not previously known to decrease blood glucose level or increase insulin secretion when it binds to the receptor. The term AGONIST also includes PARTIAL AGONISTS which are materials, for example, ligands or candidate compounds, which activate the cellular response when they bind to the receptor to a lesser degree or extent than do full agonists.

ANTAGONIST refers to material, for example, ligands or candidate compounds that competitively bind to the receptor at the same site as an agonist but which does not activate a cellular response, and can thereby inhibit a cellular response elicited by the agonist. An ANTAGONIST does not diminish the baseline cellular response in the absence of an agonist. In some embodiments, an ANTAGONIST is material not previously known to compete with an agonist to inhibit a cellular response when it binds to the receptor (for example, wherein the cellular response is GTPγS binding to membranes or to the lowering of intracellular cAMP level).

CANDIDATE COMPOUND refers to a molecule (for example, a chemical compound) that is amenable to a screening technique.

COMPOSITION refers to a material comprising at least two compounds or two components; for example, a “pharmaceutical composition” is a composition.

COMPOUND EFFICACY refers to a measurement of the ability of a compound to inhibit or stimulate receptor functionality, as opposed to solely receptor binding affinity.

CONTACT or CONTACTING refers to bringing at least two moieties together, whether in an in vitro system or an in vivo system.

DIABETES as used herein is intended to encompass the usual diagnosis of diabetes made from any method including, for example, the following list: symptoms of diabetes (e.g., polyuria, polydipsia, polyphagia) plus casual blood glucose levels of greater than or equal to 200 mg/dl, wherein casual blood glucose is defined any time of the day regardless of the timing of meal or drink consumption; or 8 hour fasting blood glucose levels of greater than or equal to 126 mg/dl; or blood glucose levels of greater than or equal to 200 mg/dl two hours following oral administration of 75 g anhydrous glucose dissolved in water. In addition, the term diabetes as used herein also includes the “pre-diabetic” state as defined by the American Diabetes Association to be a fasting blood glucose level of 100-125 mg/dl or blood glucose levels of 140-199 mg/dl two hours following oral administration of glucose. Diabetes can be precipitated by several conditions including, for example, beta cell apoptosis, pregnancy (gestational diabetes), or autoimmune destruction of beta islet cells.

EFFECTIVE AMOUNT refers to an amount of active compound or pharmaceutical composition that elicits the desired biological or medicinal response in a tissue, system, or individual that is being sought by the researcher or medical doctor or other clinician. For example, an effective dose can be an amount that can treat an insulin, related disorder. Also, for example, an effective dose can be an amount that can prevent an insulin-related disorder.

GLYCEMIC MARKER refers to a molecule whose level can be correlated with insulin or blood glucose levels. For example, a glycemic marker can be glucose, insulin, or any molecule that can be correlated with glucose or insulin levels in the blood. Several assays are known in the art for determining the level of a glycemic marker such as, for example, measuring blood or urine glucose levels in a normal or genetically altered animal, measuring insulin release in a cell culture system or in an animal, and performing an oral glucose tolerance test (ogtt) in an animal.

GLYCEMIC STABILIZING COMPOUND refers to a compound that directly and acutely stabilizes blood glucose levels. Direct and acute stabilization occurs within less than 24 hours of compound use. For example, a glycemic-stabilizing compound can stabilize blood glucose levels in an individual by inhibiting glucose excursion or increasing insulin secretion in response to glucose within less than 24 hours of compound administration. The primary mode of action of a glycemic stabilizing compound as used herein is not dependent on weight loss, although weight loss may or may not be a longer term effect of the compound use.

IMPAIRED GLUCOSE TOLERANCE (IGT) as used herein is intended to indicate that condition associated with insulin-resistance that is intermediate between type 2 diabetes and normal glucose tolerance (NGT). IGT is diagnosed by a procedure wherein an affected person's postprandial glucose response is determined to be abnormal as assessed by 2-hour postprandial plasma glucose levels. In this test, a measured amount of glucose is given to the patient and blood glucose levels are measured at regular intervals, usually every half hour for the first two hours and every hour thereafter. In a “normal” or non-IGT individual, glucose levels rise during the first two hours to a level less than 140 mg/dl and then drop rapidly. In an IGT individual, the blood glucose levels are higher and the drop-off level is at a slower rate.

INDIVIDUAL refers to any animal, including mammals, preferably mice, rats, other rodents, rabbits, dogs, cats, swine, cattle, sheep, horses, or primates, and most preferably humans.

INHIBIT or INHIBITING, in relationship to the term “response” means that a response is decreased or prevented in the presence of a compound as compared to in the absence of the compound.

INSULIN-RELATED DISORDER refers to a disorder related to the level of insulin in the blood or at an organ or tissue. As used herein, an insulin-related disorder can be the result of, for example, too little insulin secretion, too much insulin secretion, or even normal insulin secretion coupled with resistance of an organ to insulin. An insulin-related disorder is intended to include, for example, a disorder that would benefit from a decrease in insulin secretion, for example, hypoglycemia, an insulinoma, a tumor where insulin is a growth factor, or aging. In addition, an insulin-related disorder is intended to include, for example, a disorder that results in elevated blood glucose and would benefit from an increase in insulin secretion. Such disorders include, for example, insulin resistance, impaired glucose tolerance or diabetes such as Type I diabetes or Type 2 diabetes. Further, in some embodiments, the term insulin-related disorder can include diseases that are related to an elevated blood glucose level, for example, atherosclerosis, heart disease, stroke, hypertension, Syndrome X, obesity, and peripheral vascular disease.

INSULIN RESISTANCE as used herein is intended to encompass the usual diagnosis of insulin resistance made by any of a number of methods, including but not restricted to: the intravenous glucose tolerance test or measurement of the fasting insulin level. It is well known that there is a good correlation between the height of the fasting insulin level and the degree of insulin resistance. Therefore, one could use elevated fasting insulin levels as a surrogate marker for insulin resistance for the purpose of identifying which normal glucose tolerance (NGT) individuals have insulin resistance. A diagnosis of insulin resistance can also be made using the euglycemic glucose clamp test.

MODULATE or MODULATION refers to an increase or decrease in the amount, quality, response or effect of a particular activity, function or molecule. DIRECTLY MODULATED, in reference to a glycemic marker, means that the marker increases or decreases the amount, quality, response or effect of a glycemic marker, within less than 24 hours. For example, a compound of the invention can increase insulin secretion from pancreatic islet cells or reduce blood glucose within minutes to hours after administration without the need to wait days for a compound to affect body weight.

PHARMACEUTICAL COMPOSITION means a composition comprising at least one compound and a pharmaceutically acceptable carrier. For example, a pharmaceutical composition can comprise at least one active ingredient, whereby the composition is amenable to investigation for a specified, efficacious outcome in an animal (for example, a mammal such as a human). Those of ordinary skill in the art will understand and appreciate the techniques appropriate for determining whether an active ingredient has a desired efficacious outcome based upon the needs of the artisan.

PREVENTING in reference to a disorder means prevention of the occurrence or onset of one or more symptoms associated with a particular disorder and does not necessarily mean the complete prevention of a disorder.

5-HT₆ RECEPTOR refers to a polypeptide with the amino acid sequence as shown in SEQ ID NO:2 (human 5-HT₆ receptor), or a variant or ortholog of this sequence that retains substantially the function of a polypeptide with the amino acid sequence as referenced in SEQ ID NO: 2.

5-HT₆ RECEPTOR FUNCTIONALITY refers to the ability of the 5-HT₆ receptor to receive a stimulus and moderate an effect in the cell, including, for example, effecting a catalytic reaction, and/or modulating activity through G-proteins. A 5-HT₆ functionality can be, for example, binding a G-protein such as G alpha s, signaling through a second messenger such as cAMP or IP3 (when using a chimeric G-protein), specifically binding to a 5HT-6-specific antibody, specifically binding to a compound such as a 5-HT₆ agonist, modulating insulin secretion or modulating blood glucose levels in vivo.

SECOND MESSENGER means an intracellular response produced as a result of receptor activation. A second messenger can include, for example, inositol triphosphate (IP3), diacylglycerol (DAG), cyclic AMP (cAMP), cyclic GMP (cGMP), and Ca²⁺. Second messenger response can be measured for a determination of receptor activation. In addition, second messenger response can be measured for the direct identification of candidate compounds, including for example, inverse agonists, partial agonists, agonists, and antagonists.

TREATING in reference to a disorder means a reduction in severity of one or more symptoms associated with a particular disorder. Therefore, treating a disorder does not necessarily mean a reduction in severity of all symptoms associated with a disorder and does not necessarily mean a complete reduction in the severity of one or more symptoms associated with a disorder.

The invention provides a method for identifying a glycemic stabilizing compound, comprising: a) contacting a candidate compound with a 5-HT₆ receptor, b) determining whether 5-HT₆ receptor functionality is modulated, and c) determining whether a glycemic marker is directly modulated, wherein a modulation in 5-HT₆ receptor functionality and direct modulation of a glycemic marker identifies the candidate compound as said glycemic stabilizing compound.

In addition, the invention provides a method for identifying a glycemic stabilizing compound, comprising: a) contacting a candidate compound with a 5-HT₆ receptor, and b) determining whether 5-HT₆ receptor functionality is increased, wherein an increase in 5-HT₆ receptor functionality identifies the candidate compound as said glycemic stabilizing compound.

The invention also provides a method for identifying a glycemic stabilizing compound, comprising: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

As used herein, “5-HT₆ receptor” refers to a polypeptide with the amino acid sequence as shown in SEQ ID NO:2 (human 5-HT₆ receptor), or a variant or ortholog of this sequence that retains substantially the function of a polypeptide with the amino acid sequence as referenced in SEQ ID NO:2.

It is understood that limited variations or modifications to the 5-HT₆ receptor can be made without destroying its function. For example, the 5-HT₆ receptor is intended to include other 5-HT₆ receptor polypeptides, for example, mammalian species orthologs of the human 5-HT₆ receptor polypeptide. The sequences of species orthologs of the human 5-HT₆ receptor are present in the database, for example, a mouse ortholog of the 5-HT₆ receptor can be found in GenBank at Accession No. NP_{—}067333 and a rat ortholog of 5-HT₆ can be found in GenBank at Accession No. NP_{—}077341. In addition, the 5-HT₆ receptor includes variants such as allelic variants, splice variants and conservative amino acid substitution variants of the 5-HT₆ receptor. For example, the 5-HT₆ receptor includes variants that retain substantially the function of the wild-type 5-HT₆ receptor polypeptide such as, for example, the ability to signal through G-alpha s, the ability to specifically bind to a 5-HT₆ receptor-specific antibody, the ability to specifically bind to a compound such as a known ligand or agonist, or the ability to regulate insulin secretion or blood glucose levels. A 5-HT₆ receptor variant need not function to the same level as the wild-type 5-HT₆ receptor, and need not contain every function of the wild-type 5-HT₆ receptor.

Conservative and non-conservative amino acid changes, gaps, and insertions to an amino acid sequence can be compared to a reference sequence using available algorithms and programs such as the Basic Local Alignment Search Tool (“BLAST”) using default settings [See, e.g., Karlin and Altschul, Proc Natl Acad Sci USA (1990) 87:2264-8; Altschul et al., J Mol Biol (1990) 215:403-410; Altschul et al., Nature Genetics (1993) 3:266-72; and Altschul et al., Nucleic Acids Res (1997) 25:3389-3402].

It is understood that a fragment of the 5-HT₆ receptor which retains substantially a function of the entire polypeptide is included in the definition. For example, a signal generating domain of the 5-HT₆ receptor or a compound binding domain of the 5-HT₆ receptor can be used in lieu of the entire polypeptide. In addition, the 5-HT₆ receptor can contain heterologous sequences such as an epitope tag or other fused polypeptide. Further, the 5-HT₆ receptor can contain a label, for example, a radiolabel, fluorescent label or enzymatic label.

In one embodiment, the methods of the invention can be applied using a polypeptide comprising 99%, 98%, 97%, 96%, 95%, 94%, 93%, 92%, 91%, 90%, 85%, 80%, or 75% sequence identity to SEQ ID NO:2.

In one embodiment, said variant of the 5-HT₆ receptor is a non-endogenous, constitutively activated mutant of the 5-HT₆ receptor. In one embodiment, the 5-HT₆ receptor is derived from a mammal. In another embodiment, the 5-HT₆ receptor is human. In a further embodiment, the 5-HT₆ receptor is mouse. In one embodiment, the 5-HT₆ receptor is recombinant.

In the methods of the invention, in one embodiment, the candidate compound is not an antibody or antigen-binding derivative thereof. In another embodiment, the candidate compound is not a peptide. In a further embodiment, said candidate compound is not a polypeptide. In the methods of the invention, in one embodiment, the candidate compound specifically excludes any compound already known in the literature to modulate the 5-HT₆ receptor, for example, a known agonist or partial agonist of the 5-HT₆ receptor.

The contacting step can occur in vivo or in vitro. In one embodiment, the candidate compound is contacted with a 5-HT₆ receptor in an isolated cell. In another embodiment, the contacting step comprises contacting in vitro with a cell or cell membrane that naturally expresses the 5-HT₆ receptor GPCR. In one embodiment, the contacting step comprises contacting in vitro with a host cell or with membrane of a host cell that expresses the 5-HT₆ receptor GPCR, wherein the host cell comprises an expression vector comprising a polynucleotide encoding the receptor. In one embodiment, the contacting step is carried out in the presence of a known agonist of the GPCR.

In the methods of the invention, a control reaction can be performed to show specificity of the response. For example, mock-transfected cells can be compared to 5-HT₆ receptor transfected cells to show specificity of a response to the 5-HT₆ receptor.

In one embodiment, the method further comprises the step of comparing the modulation of the receptor caused by the candidate compound to a second modulation of the receptor caused by contacting the receptor with a known modulator of the receptor. In one embodiment, said known modulator is an agonist or partial agonist.

In the methods of the invention, determining whether 5-HT₆ receptor functionality is increased can comprise using a second messenger assay. The initiation of an intracellular signal can be determined, for example, through the measurement of the level of a second messenger such as cyclic AMP (cAMP), cyclic GMP (cGMP), inositol triphosphate (IP3), diacylglycerol (DAG), MAP kinase, or calcium. Several assays are well known in the art for measuring these second messengers, for example, cAMP assays, IP3 assays, the FLIPR assay, the melanophore assay, or CRE-reporter assay. In addition, examples of second messenger assays are disclosed herein in Examples 11-16.

In one embodiment, the step of determining whether 5-HT₆ receptor functionality is increased comprises measurement of GTPγS binding to membrane comprising the 5-HT₆ receptor, for example, using GTPγS labeled with [³⁵S]. In another embodiment, the determining step is through the measurement of the level of a second messenger selected from the group consisting of cyclic AMP (cAMP), cyclic GMP (cGMP), inositol triphosphate (IP3), diacylglycerol (DAG), MAP kinase activity, and Ca²⁺. In one embodiment, the second messenger is cAMP. In another embodiment, the measurement of cAMP is carried out using a whole-cell adenylyl cyclase assay. In a further embodiment, the measurement of cAMP is carried out with membrane comprising the 5-HT₆ receptor. In one embodiment, the determining step is through measurement of intracellular IP3, for example, including the use of a chimeric G-protein such as a Gq/Gi chimera. In another embodiment, the determining step is through a cyclic AMP reporter element (CRE)-reporter assay. Such a reporter assay can use, for example, luciferase or β-galactosidase as a reporter. In one embodiment, the determining step is through measurement of intracellular Ca²⁺. In another embodiment, the determining step is through the use of a melanophore assay.

Several methods are known in the art for determining whether a glycemic marker is directly modulated. In one embodiment, the step of determining whether a glycemic marker is directly modulated is through measuring blood or urine glucose levels. In another embodiment, the step of determining whether a glycemic marker is directly modulated is through measuring insulin release in a cell culture system or in an animal. In a further embodiment, the step of determining whether a glycemic marker is directly modulated is through performing an oral glucose tolerance test (ogtt) in an animal. Other methods of determining whether a glycemic marker is directly modulated are known in the art and include, for example, measurement of glucose uptake by adipocytes obtained from a mammal or measurement of glucose uptake by skeletal muscle cells obtained from a mammal. Further methods of determining whether a glyceminc marker is directly modulated include, for example, measurement of plasma incretin GLP-1 and glucose-dependent insulinotropic peptide (GIP) levels, or plasma PYY levels.

In one embodiment, the glycemic stabilizing compound decreases blood glucose concentration. In another embodiment, the glycemic stabilizing compound increases insulin secretion. In a further embodiment, the glycemic stabilizing compound regulates insulin concentration in the blood.

In one embodiment, said glycemic stabilizing compound is a 5-HT₆ receptor agonist or partial agonist. In one embodiment, an EC50 value for the 5-HT₆ receptor agonist or partial agonist is determined using an assay selected from the group consisting of: a cAMP assay carried out using transfected cells expressing recombinant 5-HT₆ receptor polypeptide; and a melanophore assay carried out using transfected melanophores expressing recombinant 5-HT₆ receptor polypeptide. In one embodiment, said glycemic stabilizing compound is a 5-HT₆ receptor agonist or partial agonist with an EC50 of less than 10 μM, of less than 1 μM, of less than 100 nM, or of less than 10 nM. In one embodiment, said glycemic stabilizing compound is a 5-HT₆ receptor agonist or partial agonist with an EC50 of less than 10 μM, of less than 9 μM, of less than 8 μM, of less than 7 μM, of less than 6 μM, of less than 5 μM, of less than 4 μM, of less than 3 μM, of less than 2 μM, of less than 1 μM, of less than 900 nM, of less than 800 nM, of less than 700 nM, of less than 600 nM, of less than 500 nM, of less than 400 nM, of less than 300 nM, of less than 200 nM, of less than 100 nM, of less than 90 nM, of less than 80 nM, of less than 70 nM, of less than 60 nM, of less than 50 nM, of less than 40 nM, of less than 30 nM, of less than 20 nM, of less than 10 nM in a second messenger assay. In one embodiment, said glycemic stabilizing compound is a 5-HT₆ receptor agonist or partial agonist with an EC50 of a value selected from the interval of 1 nM to 10 μM. In another embodiment, said glycemic stabilizing compound is a 5-HT₆ receptor agonist or partial agonist with an EC50 of a value selected from the interval of 1 nM to 1 μM. In a further embodiment, said glycemic stabilizing compound is a 5-HT₆ receptor agonist or partial agonist with an EC50 of a value selected from the interval of 1 nM to 100 nM. In a yet further embodiment, said glycemic stabilizing compound is a 5-HT₆ receptor agonist or partial agonist with an EC50 of a value selected from the interval of 1 nM to 10 nM. In one embodiment, said glycemic stabilizing compound is selective for the 5-HT₆ receptor compared to other GPCRs.

In one embodiment, said glycemic stabilizing compound is orally bioavailable. In one embodiment, said oral bioavailability is at least 1%, at least 5%, at least 10%, at least 15%, at least 20%, at least 25%, at least 30%, at least 35%, at least 40%, or at least 45% relative to intraperitoneal administration. In one embodiment, said orally bioavailable glycemic stabilizing compound is able to cross the blood-brain barrier. In another embodiment, said orally bioavailable glycemic stabilizing compound is not able to cross the blood-brain barrier.

The invention provides a method for identifying a glycemic stabilizing compound, comprising: a) contacting a candidate compound with a 5-HT₆ receptor, b) determining whether 5-HT₆ receptor functionality is modulated, and c) determining whether a glycemic marker is directly modulated, wherein the modulation of the glyceimic marker is first detected within less than 24 hours of the contacting of the candidate compound with the 5-HT₆ receptor, and wherein a modulation in 5-HT₆ receptor functionality and direct modulation of a glycemic marker identifies the candidate compound as said glycemic stabilizing compound.

The invention provides a method for identifying a glycemic stabilizing compound, comprising: a) contacting a candidate compound with a 5-HT₆ receptor, b) determining whether 5-HT₆ receptor functionality is modulated, and c) determining whether a glycemic marker is directly modulated, wherein the modulation of the glycemic marker is first detected within less than 24 hours of the contacting of the candidate compound with the 5-HT₆ receptor, and wherein a modulation in 5-HT₆ receptor functionality and direct modulation of a glycemic marker identifies the candidate compound as said glycemic stabilizing compound.

The invention also provides a method for identifying a glycemic stabilizing compound, comprising: a) contacting a candidate compound with a 5-HT₆ receptor, b) determining whether 5-HT₆ receptor functionality is increased, and c) determining whether a glycemic marker is directly modulated, wherein the modulation of the glycemic marker is first detected within less than 24 hours of the contacting of the candidate compound with the 5-HT₆ receptor, and wherein an increase in 5-HT₆ receptor functionality and direct modulation of a glycemic marker identifies the candidate compound as said glycemic stabilizing compound. In one embodiment, the modulation of the glycemic marker is first detected within less than 12 hours of the contacting of the candidate compound with the 5-HT₆ receptor. In another embodiment, the modulation of the glycemic marker is first detected within less than 6 hours of the contacting of the candidate compound with the 5-HT₆ receptor. In a further embodiment, the modulation of the glycemic marker is first detected within less than 4 hours of the contacting of the candidate compound with the 5-HT₆ receptor. In a yet further embodiment, the modulation of the glycemic marker is first detected within less than 2 hours of the contacting of the candidate compound with the 5-HT₆ receptor. In another embodiment, the modulation of the glycemic marker is first detected within less than 1 hour of the contacting of the candidate compound with the 5-HT₆ receptor. In a further embodiment, the modulation of the glycemic marker is first detected within less than 30 minutes of the contacting of the candidate compound with the 5-HT₆ receptor. In a yet further embodiment, the modulation of the glycemic marker is first detected within less than 10 minutes of the contacting of the candidate compound with the 5-HT₆ receptor. In another embodiment, the modulation of the glycemic marker is first detected within less than 1 minute of the contacting of the candidate compound with the 5-HT₆ receptor.

In one embodiment, said 5-HT₆ receptor is recombinant. In another embodiment, said contacting comprises contacting with a host cell or with membrane of a host cell that expresses the 5-HT₆ receptor, wherein the host cell comprises an expression vector comprising a polynucleotide encoding the receptor. In a further embodiment, said 5-HT₆ receptor is human.

In one embodiment, said determining comprises a second messenger assay. In another embodiment, said glycemic stabilizing compound decreases blood glucose concentration. In a further embodiment, said glycemic stabilizing compound increases insulin secretion. In one embodiment, said glycemic marker is glucose and in another embodiment said glycemic marker is insulin.

The invention also provides a method for identifying a glycemic stabilizing compound, comprising: a) contacting a single dose of a candidate compound with a 5-HT₆ receptor, b) determining whether 5-HT₆ receptor functionality is modulated, and c) determining whether a glycemic marker is directly modulated, wherein a modulation in 5-HT₆ receptor functionality and direct modulation of a glycemic marker identifies the candidate compound as said glycemic stabilizing compound.

The invention further provides a method for identifying a glycemic stabilizing compound, comprising: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

A single dose of a candidate compound can be used in the methods of the invention. For example, a single dose of a candidate compound can be applied to an isolated cell or cell membrane in a second messenger assay. For example, a single dose of a candidate compound can be added to an isolated cell or cell membrane in a high through-put cAMP assay. In addition, for example, a single dose of a candidate compound can be given to an animal such as a mouse for use in an assay such as an oGTT (see Example 5 herein where a dose of 1.0 mpk, 3.3 mpk, and 10 mpk of a known compound were given to different C57/Bl6 mice for an oGTT).

In one embodiment, said 5-HT₆ receptor is recombinant. In another embodiment, said contacting comprises contacting with a host cell or with membrane of a host cell that expresses the 5-HT₆ receptor, wherein the host cell comprises an expression vector comprising a polynucleotide encoding the receptor. In a further embodiment, said 5-HT₆ receptor is human.

In one embodiment, said determining comprises a second messenger assay. In another embodiment, said glycemic stabilizing compound decreases blood glucose concentration. In a further embodiment, said glycemic stabilizing compound increases insulin secretion. In one embodiment, said glycemic marker is glucose and in another embodiment said glycemic marker is insulin.

The invention provides a method for identifying a glycemic stabilizing compound, comprising: a) contacting a candidate compound with a 5-HT₆ receptor in an isolated cell, b) determining whether 5-HT₆ receptor functionality is modulated, and c) determining whether a glycemic marker is directly modulated, wherein a modulation in 5-HT₆ receptor functionality and direct modulation of a glycemic marker identifies the candidate compound as said glycemic stabilizing compound.

The invention also provides a method for identifying a glycemic stabilizing compound, comprising: a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

An isolated cell used in the method can be a primary cell or a transformed cell, for example, a cell line. The 5-HT₆ receptor can be naturally expressed in the isolated cell or the cell can be engineered to express the receptor. Several techniques are well known in the art for expressing a receptor in an isolated cell. In one embodiment, said isolated cell is a cell line that has been transfected with a 5-HT₆ receptor nucleic acid such that the cell expresses the 5-HT₆ receptor on its surface. Isolated cells are useful in screening assay techniques as described herein and known in the art.

In one embodiment, said 5-HT₆ receptor is recombinant. In another embodiment, said contacting comprises contacting with a host cell or with membrane of a host cell that expresses the 5-HT₆ receptor, wherein the host cell comprises an expression vector comprising a polynucleotide encoding the receptor. In a further embodiment, said 5-HT₆ receptor is human.

In one embodiment, said determining comprises a second messenger assay. In another embodiment, said glycemic stabilizing compound decreases blood glucose concentration. In a further embodiment, said glycemic stabilizing compound increases insulin secretion. In one embodiment, said glycemic marker is glucose and in another embodiment said glycemic marker is insulin.

The invention provides a method for treating or preventing an insulin-related disorder in an individual in need thereof, comprising administering to said individual an effective amount of the glycemic stabilizing compound identified by the methods of the invention described herein. For example, the invention provides a method for treating or preventing an insulin-related disorder in an individual in need thereof, comprising administering to said individual an effective amount of the glycemic stabilizing compound identified by: a) contacting a candidate compound with a 5-HT₆ receptor, b) determining whether 5-HT₆ receptor functionality is modulated, and c) determining whether a glycemic marker is directly modulated, wherein a modulation in 5-HT₆ receptor functionality and direct modulation of a glycemic marker identifies the candidate compound as said glycemic stabilizing compound.

For example, in one embodiment, the invention provides a method for treating or preventing an insulin-related disorder in an individual in need thereof, comprising administering to said individual an effective amount of the glycemic stabilizing compound identified by: a) contacting a candidate compound with a 5-HT₆ receptor, and b) determining whether 5-HT₆ receptor functionality is increased, wherein an increase in 5-HT₆ receptor functionality identifies the candidate compound as said glycemic stabilizing compound.

In another embodiment, the invention provides a method for treating or preventing an insulin-related disorder in an individual in need thereof, comprising administering to said individual an effective amount of the glycemic stabilizing compound identified by: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In one embodiment, the method further comprising administering to said individual an effective amount of an agent used for the treatment of diabetes, blood lipid disorders, or obesity in combination with an effective amount of the glycemic stabilizing compound. In another embodiment, said insulin-related disorder is insulin resistance, impaired glucose tolerance, or diabetes. In a further embodiment, the individual is a human.

In another embodiment, the invention provides a method for treating or preventing an insulin-related disorder in an individual in need thereof, comprising administering to said individual an effective amount of the glycemic stabilizing compound identified by: a) contacting a candidate compound with a 5-HT₆ receptor, b) determining whether 5-HT₆ receptor functionality is increased, and c) determining whether a glycemic marker is directly modulated, wherein the modulation of the glycemic marker is first detected within less than 24 hours of the contacting of the candidate compound with the 5-HT₆ receptor, and wherein an increase in 5-HT₆ receptor functionality and direct modulation of a glycemic marker identifies the candidate compound as said glycemic stabilizing compound.

In one embodiment, the method further comprising administering to said individual an effective amount of an agent used for the treatment of diabetes, blood lipid disorders, or obesity in combination with an effective amount of the glycemic stabilizing compound. In another embodiment, said insulin-related disorder is insulin resistance, impaired glucose tolerance, or diabetes. In a further embodiment, the individual is a human.

The invention provides a method for treating or preventing an insulin-related disorder in an individual in need thereof, comprising administering to said individual an effective amount of the glycemic stabilizing compound by: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In one embodiment, the method further comprising administering to said individual an effective amount of an agent used for the treatment of diabetes, blood lipid disorders, or obesity in combination with an effective amount of the glycemic stabilizing compound. In another embodiment, said insulin-related disorder is insulin resistance, impaired glucose tolerance, or diabetes. In a further embodiment, the individual is a human.

The invention provides a method for treating or preventing an insulin-related disorder in an individual in need thereof, comprising administering to said individual an effective amount of the glycemic stabilizing compound identified by: a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In one embodiment, the method further comprising administering to said individual an effective amount of an agent used for the treatment of diabetes, blood lipid disorders, or obesity in combination with an effective amount of the glycemic stabilizing compound. In another embodiment, said insulin-related disorder is insulin resistance, impaired glucose tolerance, or diabetes. In a further embodiment, the individual is a human.

In one embodiment, said insulin-related disorder is hypoglycemia, an insulin-secreting or insulin-dependent tumor, aging, insulin resistance, impaired glucose tolerance, or diabetes. In another embodiment, said insulin-related disorder is insulin resistance, impaired glucose tolerance or diabetes. In a further embodiment, said insulin-related disorder is diabetes. In a yet further embodiment, said insulin-related disorder is type 2 diabetes. In another embodiment, said insulin-related disorder includes a condition related to an elevated blood glucose concentration, such as atherosclerosis, heart disease, stroke, hypertension, obesity, Syndrome X or peripheral vascular disease.

In the methods of treatment or prevention of an insulin-related disorder in an individual, the individual would be one in need of treatment or prevention of an insulin-related disorder. This would be a judgment made by a caregiver (e.g. physician, nurse, nurse practitioner, etc. in the case of humans; veterinarian in the case of animals, including non-human mammals) that an individual or animal requires or will benefit from treatment. This judgment is made based on a variety of factors that are in the realm of a caregiver's expertise, including the knowledge that the individual or animal is ill, or will be ill, as the result of a condition that is treatable by the compounds of the invention. Treating can also encompass slowing or stopping the progression of a disorder.

Diabetes and related conditions such as insulin resistance and impaired glucose tolerance have been described herein.

In addition, insulin resistance is a common feature of polycystic ovary syndrome (PCOS) and drugs such as rosiglitazone and metformin has been used in the treatment of PCOS (Sepilian and Nagamani J. Clin. Endocrinol Metab. Oct. 14, 2003; Baillargeon et al., Fertil. Steril. 82:893-902 (2004)). PCOS is characterized, for example, by bilaterally enlarged polycystic ovaries, amenorrhea, and infertility. It is inherited as an autosomal dominant condition. Other symptoms of the disease can include, for example, hirsutism and obesity. Hormonally, PCOS is characterized, for example, by increased secretion of leutinizing hormone, insulin and androgens.

Another indication for the compounds of the invention is treatment of lipodystrophy, for example, as caused by anti-retroviral therapy for HIV infection. Some patients on long term AIDS therapy known as highly active anti-retroviral therapy (HAART) are increasingly developing a syndrome called lipodystrophy. Symptoms include insulin sensitivity, the redistribution of fat from the face, arms and legs to the abdomen and upper back, and cholesterol changes. About 14 percent of people on HAART eventually develop type 2 diabetes. The drug rosiglitazone has been shown to improve insulin sensitivity in HIV-positive patients who received the treatment for three months. Patients had about a 20 percent improvement on a standard test to measure insulin sensitivity and also increased their total body fat, particularly the amount of fat on their face, arms and legs, which went up by 24 percent. By comparison, patients taking the placebo had a 2 percent decrease in face, arm and leg fat.

In one embodiment, said insulin-related disorder includes a condition related to an elevated blood glucose concentration, such as atherosclerosis, heart disease, stroke, hypertension, obesity, Syndrome X and peripheral vascular disease.

Atherosclerosis is a process where deposits of fatty substances, cholesterol and other substances build up in the inner lining of an artery. This buildup is called plaque. Plaques that rupture cause blood clots to form that can block blood flow to the heart (heart attack) or the brain (stroke). Heart attack is the number one cause of death for both men and women in the United States and stroke is the number three cause of death [see, for example, Nature Medicine, Special Focus on Atherosclerosis, (2002) 8:1209-1262]. Abnormally high levels of circulating lipids are a major predisposing factor in development of atherosclerosis. Elevated levels of low density lipoprotein (LDL) cholesterol, elevated levels of triglycerides, or low levels of high density lipoprotein (HDL) cholesterol are, independently, risk factors for atherosclerosis and associated pathologies.

Heart disease includes, but is not limited to, cardiac insufficiency, coronary insufficiency, coronary artery disease, and high blood pressure (hypertension). Peripheral vascular disease refers to diseases of blood vessels outside the heart and brain. Organic peripheral vascular diseases are caused by structural changes in the blood vessels, such as inflammation and tissue damage. Peripheral artery disease is an example. Peripheral artery disease (PAD) is a condition similar to coronary artery disease and carotid artery disease. In PAD, fatty deposits build up along artery walls and affect blood circulation, mainly in arteries leading to the legs and feet. In its early stages a common symptom is cramping or fatigue in the legs and buttocks during activity. Such cramping subsides when the person stands still. This is called “intermittent claudication.” People with PAD have a higher risk of death from stroke and heart attack, due to the risk of blood clots.

Syndrome X, also called metabolic syndrome, is characterized by a group of metabolic risk factors in one person. They include: central obesity (excessive fat tissue in and around the abdomen), atherogenic dyslipidemia (blood fat disorders—mainly high triglycerides and low HDL cholesterol), raised blood pressure (130/85 mmHg or higher), insulin resistance or glucose intolerance, prothrombotic state (e.g., high fibrinogen or plasminogen activator inhibitor [−1] in the blood), and proinflammatory state (e.g., elevated high-sensitivity C-reactive protein in the blood).

While the compounds identified by the methods of the invention can be administered as the sole active pharmaceutical agent (preferably as formulated into a pharmaceutical composition), they can also be used in combination with one or more agents including, for example, agents that are used for the treatment of diabetes, blood lipid disorders, or obesity. For example, a compound such as a 5-HT₆ receptor agonist or partial agonist can be used in combination with one or more agents belonging to the class of drugs known as α-glucosidase inhibitors, aldose reductase inhibitors, biguanides, thiazolidinediones, meglitinides, sulfonylureas, incretin mimetics, insulin, HMG-CoA reductase inhibitors, squalene synthesis inhibitors, fibrate compounds, LDL catabolism enhancers, angiotensin converting enzyme (ACE) inhibitors, lipase inhibitors, serotonin and/or noradrenaline releasers or reuptake inhibitors.

α-Glucosidase inhibitors belong to the class of drugs which competitively inhibit digestive enzymes such as α-amylase, maltase, α-dextrinase, sucrase, etc. in the pancreas and or small intestine. The reversible inhibition by α-glucosidase inhibitors retard, diminish or otherwise reduce blood glucose levels by delaying the digestion of starch and sugars. Some representative examples of α-glucosidase inhibitors include acarbose, N-(1,3-dihydroxy-2-propyl)valiolamine (generic name; voglibose), miglitol, and α-glucosidase inhibitors known in the art.

Aldose reductase inhibitors are drugs which inhibit the first-stage rate-limiting enzyme in the polyol pathway and thereby prevent or arrest diabetic complications. In the hyperglycemic state of diabetes, the utilization of glucose in the polyol pathway is increased and the excess sorbitol accumulated intracellularly as a consequence acts as a tissue toxin and hence evokes the onset of complications such as diabetic neuropathy, retinopathy, and nephropathy. Examples of the aldose reductase inhibitors include tolurestat; epalrestat; 3,4-dihydro-2,8-diisopropyl-3-thioxo-2H-1,4-benzoxazine-4-acetic acid; 2,7-difluorospiro(9H-fluorene-9,4′-imidazolidine)-2′,5′-dione (generic name: imirestat); 3-[(4-bromo-2-fluorophenyl)methy]-7-chloro-3,4-dihydro-2,4-dioxo-1(2H)-quinazoline acetic acid (generic name: zenarestat); 6-fluoro-2,3-dihydro-2′,5′-dioxo-spiro[4H-1-benzopyran-4,4′-imidazolidine]-2-carboxamide (SNK-860); zopolrestat; sorbinil; and 1-[(3-bromo-2-benzofuranyl)sulfonyl]-2,4-imidazolidinedione (M-16209), and aldose reductase inhibitors known in the art.

The biguanides are a class of drugs that stimulate anaerobic glycolysis, increase the sensitivity to insulin in the peripheral tissues, inhibit glucose absorption from the intestine, suppress of hepatic gluconeogenesis, and inhibit fatty acid oxidation. Examples of biguanides include phenformin, metformin, buformin, and biguanides known in the art.

Insulin secretion enhancers belong to the class of drugs having the property to promote secretion of insulin from pancreatic β cells. Examples of the insulin secretion enhancers include sulfonylureas (SU). The sulfonylureas (SU) are drugs which promote secretion of insulin from pancreatic β cells by transmitting signals of insulin secretion via SU receptors in the cell membranes. Examples of the sulfonylureas include tolbutamide; chlorpropamide; tolazamide; acetohexamide; 4-chloro-N-[(1-pyrrolidinylamino) carbonyl]-benzenesulfonamide (generic name: glycopyramide) or its ammonium salt; glibenclamide (glyburide); gliclazide; 1-butyl-3-metanilylurea; carbutamide; glibonuride; glipizide; gliquidone; glisoxepid; glybuthiazole; glibuzole; glyhexamide; glymidine; glypinamide; phenbutamide; tolcyclamide, glimepiride, and other insulin secretion enhancers known in the art. Other insulin secretion enhancers include N-[[4-(1-methylethyl)cyclohexyl)carbonyl]-D-phenylalanine (Nateglinide); calcium (2S)-2-benzyl-3-(cis-hexahydro-2-isoindolinylcarbonyl)propionate dihydrate (Mitiglinide, KAD-1229); and other insulin secretion enhancers known in the art.

Thiazolidinediones belong to the class of drugs more commonly known as TZDs. Thiazolidinediones are a class of drugs for type 2 diabetes that lower the blood sugar by increasing the sensitivity of cells to insulin. Insulin can then move glucose from the blood into cells for energy. These drugs can also increase HDL.

Examples of thiazolidinediones include rosiglitazone, pioglitazone, and thiazolidinediones known in the art. Rezulin (troglitazone) was the first drug in this class in the U.S., but was taken off the market because of liver toxicity. Sister compounds now available with a better safety profile include Actos (pioglitazone) and Avandia (rosiglitazone). The main contraindications to the use of these medications include liver disease and heart failure. These drugs can also cause a significant increase in fluid retention and thereby increase the risk of heart failure.

Meglitinides are used to stop the rapid rise in blood sugar that can occur immediately after a person with type 2 diabetes eats a meal. These compounds, which include, for example, repaglinide (Prandin) and nateglinide (Starlix), work by increasing the amount of insulin produced by the pancreas similar to the way sulfonyurea medications work. Meglitinides are taken before eating a meal. Side effects associated with this class of drugs includes low blood sugar, upper respiratory infections including sinus conditions, headache, joint and back pain, nausea, diarrhea and constipation.

Recently, an incretin mimetic has been approved for use in diabetes treatment. The drug Byetta (exenatide) is a peptide drug derived from the Gila monster lizard. It mimics the effect of incretins such as GLP-1. The drug, which is injected, has been approved for combination therapy with other approved diabetes drugs.

The different types of insulin are categorized according to how fast they start to work (onset) and how long they continue to work (duration). The types now available include rapid-, short-, intermediate-, and long-acting insulin. There are premixed rapid- and intermediate-acting insulins available, including: 70% intermediate-acting (NPH) and 30% short-acting regular insulin, called 70/30 insulin; 50% intermediate-acting (NPH) and 50% short-acting regular insulin, called 50/50 insulin; 75% intermediate-acting (NPH) and 25% rapid-acting Humalog (lispro), called 75/25 insulin; 70% intermediate-acting (NPH); and 30% rapid-acting NovoLog (insulin aspart), called NovoLog Mix 70/30. Insulin usually is given as an injection into the tissues under the skin (subcutaneous). It can also be given through an insulin pump or jet injector, a device that sprays the medication into the skin, or an inhaler.

Insulin lets sugar (glucose) enter cells, where it is used for energy. Without insulin, the blood sugar level rises above what is safe for the body. Usually, a rapid- or short-acting and an intermediate- or long-acting insulin is taken to provide the constant and variable levels of insulin that the body needs. The short-acting insulin reduces blood sugar levels quickly and then wears off. Some long-acting insulins start taking effect when rapid- or short-acting insulins begin to wear off. The new long-acting insulin, Lantus, starts to work within a few minutes after it is given and continues to work at the same rate for about 24 hours.

The combination of a rapid- or short-acting and intermediate- or long-acting insulin helps keep blood sugar levels within a range that is safe for the body throughout the day. Thus insulin can be used to treat people with type 1 diabetes, people with type 2 diabetes whose pancreas produces little or no insulin or whose oral medications do not control their blood sugar. These people may take insulin either alone or along with oral medication, people with type 2 diabetes whose blood sugar levels are high because of a severe illness or major surgery, women with type 2 diabetes who are pregnant or breast-feeding who cannot keep their blood sugar levels within a safe range with diet and exercise. Only one oral diabetes medication (glyburide) has been studied for use during pregnancy.

The major side effect of insulin can be a dangerously low blood sugar level (severe hypoglycemia). A very low blood sugar level can develop within 10 to 15 minutes. Insulin can contribute to weight gain, especially in people with type 2 diabetes who already are overweight. Other possible side effects of long-term insulin use include the loss of fatty tissue (lipodystrophy) where the insulin is injected and, rarely, allergic reactions that include swelling (edema).

Statin compounds belong to a class of drugs that lower blood cholesterol levels by inhibiting hydroxymethylglutalyl CoA (HMG-CoA) reductase. HMG-CoA reductase is the rate-limiting enzyme in cholesterol biosynthesis. A statin that inhibits this reductase lowers serum LDL concentrations by upregulating the activity of LDL receptors and responsible for clearing LDL from the blood. Examples of the statin compounds include rosuvastatin, pravastatin and its sodium salt, simvastatin, lovastatin, atorvastatin, fluvastatin, cerivastatin, and HMG-CoA reductase inhibitors known in the art.

Squalene synthesis inhibitors belong to a class of drugs that lower blood cholesterol levels by inhibiting synthesis of squalene. Examples of the squalene synthesis inhibitors include (S)-α-[Bis[2,2-dimethyl-1-oxopropoxy)methoxy]phosphinyl]-3-phenoxybenzenebutanesulfonic acid, mono potassium salt (BMS-188494) and squalene synthesis inhibitors known in the art.

Fibrate compounds belong to a class of drugs that lower blood cholesterol levels by inhibiting synthesis and secretion of triglycerides in the liver and activating a lipoprotein lipase. Fibrates have been known to activate peroxisome proliferators-activated receptors and induce lipoprotein lipase expression. Examples of fibrate compounds include bezafibrate, beclobrate, binifibrate, ciplofibrate, clinofibrate, clofibrate, clofibric acid, etofibrate, fenofibrate, gemfibrozil, nicofibrate, pirifibrate, ronifibrate, simfibrate, theofibrate, and fibrates known in the art.

LDL (low-density lipoprotein) catabolism enhancers belong to a class of drugs that lower blood cholesterol levels by increasing the number of LDL (low-density lipoprotein) receptors, examples include LDL catabolism enhancers known in the art.

Angiotensin converting enzyme (ACE) inhibitors belong to the class of drugs that partially lower blood glucose levels as well as lowering blood pressure by inhibiting angiotensin converting enzymes. Examples of the angiotensin converting enzyme inhibitors include captopril, enalapril, alacepril, delapril; ramipril, lisinopril, imidapril, benazepril, ceronapril, cilazapril, enalaprilat, fosinopril, moveltopril, perindopril, quinapril, spirapril, temocapril, trandolapril, and angiotensin converting enzyme inhibitors known in the art.

Lipase inhibitors include, for example, anti-obesity compounds such as Orlistat (XENICAL™). Orlistat inhibits fat absorption directly but also tends to produce a high incidence of unpleasant gastric side-effects such as diarrhea and flatulence.

Another class of anti-obesity drugs includes serotonin and/or noradrenaline releasers or reuptake inhibitors. For example, sibutramine (Meridia™) is a mixed 5-HT/noradrenaline reuptake inhibitor. The main side effect of sibutramine can be an increase in blood pressure and heart rate in some patients. The serotonin releaser/reuptake inhibitors fenfluramine (Pondimin™) and dexfenfluramine (Redux™) have been reported to decrease food intake and body weight over a prolonged period. However, both products were withdrawn from use after reports of heart valve abnormalities associated with their use.

One embodiment of the invention includes a method of treating or preventing an insulin-related disorder in an individual in need thereof, comprising administering to the individual an effective amount of a glycemic stabilizing compound identified using one of the methods of the invention in combination with an effective amount of an agent used for the treatment of diabetes, blood lipid disorders, or obesity. In one embodiment said agent used for the treatment of diabetes, blood lipid disorder, or obesity is selected from the group consisting of an α-glucosidase inhibitor, an aldose reductase inhibitor, a biguanide, a HMG-CoA reductase inhibitor, a squalene synthesis inhibitor, a fibrate compound, a LDL catabolism enhancer and an angiotensin converting enzyme inhibitor. In another embodiment, the HMG-CoA reductase inhibitor is selected from the group consisting of prevastatin, simvastatin, lovastatin, atorvastatin, fluvastatin and lipitor.

In accordance with the present invention, the combination can be used by mixing the respective active components either all together or independently with a physiologically acceptable carrier, excipient, binder, diluent, etc., as described herein and known in the art, and administering the mixture or mixtures either orally or non-orally as a pharmaceutical composition. When a compound or a mixture of compounds are administered as a combination therapy or prophylaxis with another active compound the therapeutic agents can be formulated as a separate pharmaceutical compositions given at the same time or at different times, or the therapeutic agents can be given as a single composition. For example, the 5-HT₆ receptor modulator and the agent used for the treatment of diabetes, blood lipid disorders, or obesity can be present in a combined preparation for simultaneous, separate or sequential use.

The invention also provides a method for decreasing blood glucose levels in an individual in need thereof, comprising administering to the individual an effective amount of the glycemic stabilizing compound identified by the methods of the invention described herein. A decrease in blood glucose can be desired, for example, in individuals with hyperglycemia such as diabetics.

In one embodiment, the invention provides a method for decreasing blood glucose levels in an individual in need thereof, comprising administering to the individual an effective amount of a glycemic stabilizing compound identified by: a) contacting a candidate compound with a 5-HT₆ receptor, b) determining whether 5-HT₆ receptor functionality is modulated, and c) determining whether a glycemic marker is directly modulated, wherein a modulation in 5-HT₆ receptor functionality and direct modulation of a glycemic marker identifies the candidate compound as said glycemic stabilizing compound.

In one embodiment, the invention provides a method for decreasing blood glucose levels in an individual in need thereof, comprising administering to the individual an effective amount of a glycemic stabilizing compound identified by: a) contacting a candidate compound with a 5-HT₆ receptor, and b) determining whether 5-HT₆ receptor functionality is increased, wherein an increase in 5-HT₆ receptor functionality identifies the candidate compound as said glycemic stabilizing compound.

In another embodiment, the invention provides a method for decreasing blood glucose levels in an individual in need thereof, comprising administering to the individual an effective amount of a glycemic stabilizing compound identified by: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In a further embodiment, the invention provides a method for decreasing blood glucose levels in an individual in need thereof, comprising administering to the individual an effective amount of a glycemic stabilizing compound identified by: a) contacting a candidate compound with a 5-HT₆ receptor, b) determining whether 5-HT₆ receptor functionality is increased, and c) determining whether a glycemic marker is directly modulated, wherein the modulation of the glycemic marker is first detected within less than 24 hours of the contacting of the candidate compound with the 5-HT₆ receptor, and wherein an increase in 5-HT₆ receptor functionality and direct modulation of a glycemic marker identifies the candidate compound as said glycemic stabilizing compound.

In one embodiment, the invention provides a method for decreasing blood glucose levels in an individual in need thereof, comprising administering to the individual an effective amount of a glycemic stabilizing compound identified by: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention provides a method for decreasing blood glucose levels in an individual in need thereof, comprising administering to the individual an effective amount of a glycemic stabilizing compound identified by: a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

The invention also provides a method for increasing insulin secretion in an individual in need thereof, comprising administering to the individual an effective amount of the glycemic stabilizing compound identified by the methods of the invention described herein. An increase in insulin secretion can be desired, for example, in individuals with hyperglycemia such as diabetics. In one embodiment, the invention provides a method for increasing insulin secretion in an individual in need thereof; comprising administering to the individual an effective amount of a glycemic stabilizing compound identified by: a) contacting a candidate compound with a 5-HT₆ receptor, b) determining whether 5-HT₆ receptor functionality is modulated, and c) determining whether a glycemic marker is directly modulated, wherein a modulation in 5-HT₆ receptor functionality and direct modulation of a glycemic marker identifies the candidate compound as said glycemic stabilizing compound.

In another embodiment, the invention provides a method for increasing insulin secretion in an individual in need thereof, comprising administering to the individual an effective amount of a glycemic stabilizing compound identified by: a) contacting a candidate compound with a 5-HT₆ receptor, and b) determining whether 5-HT₆ receptor functionality is increased, wherein an increase in 5-HT₆ receptor functionality identifies the candidate compound as said glycemic stabilizing compound.

In one embodiment, the invention provides a method for increasing insulin secretion in an individual in need thereof, comprising administering to the individual an effective amount of a glycemic stabilizing compound identified by: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention provides a method for increasing insulin secretion in an individual in need thereof, comprising administering to the individual an effective amount of a glycemic stabilizing compound identified by:

a) contacting a candidate compound with a 5-HT₆ receptor, b) determining whether 5-HT₆ receptor functionality is increased, and c) determining whether a glycemic marker is directly modulated, wherein the modulation of the glycemic marker is first detected within less than 24 hours of the contacting of the candidate compound with the 5-HT₆ receptor, and wherein an increase in 5-HT₆ receptor functionality and direct modulation of a glycemic marker identifies the candidate compound as said glycemic stabilizing compound.

In a further embodiment, the invention provides a method for increasing insulin secretion in an individual in need thereof, comprising administering to the individual an effective amount of a glycemic stabilizing compound identified by: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention provides a method for increasing insulin secretion in an individual in need thereof, comprising administering to the individual an effective amount of a glycemic stabilizing compound identified by:

a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

The invention further provides a method for increasing insulin secretion in a glucose dependent manner in an individual in need thereof, comprising administering to the individual an effective amount of a 5-HT₆ receptor agonist or partial agonist. As shown herein in FIG. 5 (bottom panel), a 5-HT₆ receptor agonist increases insulin release in response to a glucose bolus. In this experiment, the 5-HT₆ receptor agonist was given to the mice 30 minutes before the addition of a glucose bolus. At time 0, there was no increase in insulin secretion, but at times after time 0 there was an increase in insulin secretion. Thus, insulin was secreted in a glucose dependent manner.

The term “in a glucose dependent manner” means that insulin secretion is increased in response to a high concentration of glucose, but not in response to a low concentration of glucose. Some drugs that have been used for the treatment of diabetes increase insulin secretion regardless of the level of glucose in the blood. This is not desirable because these drugs increase insulin secretion even under conditions of hypoglycemia. The increased insulin then further exacerbates the hypoglycemia, sometimes to critical levels. A high concentration of glucose means that the concentration of glucose in the blood or around cells is higher than a normal fasting glucose concentration range, for example, 16.8 mmol/L is a high concentration of glucose. A low concentration of glucose means that the concentration of glucose in the blood or around cells is lower than a normal fasting glucose concentration range, for example, 3.3 mmol/L or less. A normal fasting glucose concentration is about 5.0 mmol/L in an animal.

The cellular mechanism of action of insulin secretion is the increase in intracellular cAMP. As disclosed herein, the 5-HT₆ receptor is expressed in L cells in the gut. The 5-HT₆ receptor is coupled to Gs so an agonist or partial of 5-HT₆ receptor can result in an increase in cAMP in L cells and the release of GLP-1. GLP-1 can bind to its receptor on pancreatic beta cells which then results in insulin secretion from the beta cell.

The invention also relates to a method of identifying a candidate compound as a potentiator of insulin secretion, comprising a) contacting a candidate compound with 5HT-6, and b) determining whether 5-HT₆ functionality is increased, wherein an increase in 5-HT₆ functionality is indicative of the candidate compound being a potentiator of insulin secretion. For example, a compound that increases 5-HT₆ functionality, such as a 5-HT₆ agonist or partial agonist, can result in an increase in insulin secretion. An increase in insulin secretion can be desired, for example, in individuals with insulin resistance such as diabetics.

The invention also relates to a further step to the methods for identifying a glycemic stabilizing compound as disclosed comprising preparing a pharmaceutical composition by combining the identified compound with at least one pharmaceutically acceptable carrier.

For example, the invention relates to a method for preparing a composition which comprises identifying a glycemic stabilizing compound and then admixing said compound with a carrier, wherein said compound is identified by the method of: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention relates to a method for preparing a composition which comprises identifying a glycemic stabilizing compound and then admixing said compound with a carrier, wherein said compound is identified by the method of: a) contacting a candidate compound with a 5-HT₆ receptor, b) determining whether 5-HT₆ receptor functionality is increased, and c) determining whether a glycemic marker is directly modulated, wherein the modulation of the glycemic marker is first detected within less than 24 hours of the contacting of the candidate compound with the 5-HT₆ receptor, and wherein an increase in 5-HT₆ receptor functionality and direct modulation of a glycemic marker identifies the candidate compound as said glycemic stabilizing compound.

In a further embodiment, the invention relates, to a method for preparing a composition which comprises identifying a glycemic stabilizing compound and then admixing said compound with a carrier, wherein said compound is identified by the method of: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention relates to a method for preparing a composition which comprises identifying a glycemic stabilizing compound and then admixing said compound with a carrier, wherein said compound is identified by the method of: a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

A compound can be formulated into pharmaceutical compositions using techniques well known to those in the art. Suitable pharmaceutically-acceptable carriers are available to those in the art; for example, see Remington's Pharmaceutical Sciences, 16^{th} Edition, 1980, Mack Publishing Co., (Oslo et al., eds.).

While it is possible that, for use in the prophylaxis or treatment, a compound disclosed herein or identified by methods of the invention can in an alternative use be administered as a raw or pure chemical, it can be useful to present the compound or active ingredient as a pharmaceutical formulation or composition further comprising a pharmaceutically acceptable carrier.

For preparing pharmaceutical compositions from the compounds identified by the methods of the invention, the selection of a suitable pharmaceutically acceptable carrier can be either solid, liquid or a mixture of both. Solid form preparations include powders, tablets, pills, capsules, cachets, suppositories, and dispersible granules. A solid carrier can be one or more substances which can also act as diluents, flavouring agents, solubilizers, lubricants, suspending agents, binders, preservatives, tablet disintegrating agents, or an encapsulating material.

In powders, the carrier is a finely divided solid which is in a mixture with the finely divided active component. In tablets, the active component is mixed with the carrier having the necessary binding capacity in suitable proportions and compacted to the desire shape and size.

The powders and tablets can contain varying percentage amounts of the active compound. A representative amount in a powder or tablet can contain from 0.5 to about 90 percent of the active compound; however, an artisan would know when amounts outside of this range are necessary. Suitable carriers for powders and tablets are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the active compound with encapsulating material as carrier providing a capsule in which the active component, with or without carriers, is surrounded by a carrier, which is thus in association with it. Similarly, cachets and lozenges are included. Tablets, powders, capsules, pills, cachets, and lozenges can be used as solid forms suitable for oral administration.

For preparing suppositories, a low melting wax, such as an admixture of fatty acid glycerides or cocoa butter, is first melted and the active component is dispersed homogeneously therein, as by stirring. The molten homogenous mixture is then poured into convenient sized molds, allowed to cool, and thereby to solidify.

Formulations suitable for vaginal administration can be presented as pessaries, tampons, creams, gels, pastes, foams or sprays containing in addition to the active ingredient such carriers as are known in the art to be appropriate.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions can be formulated according to the known art using suitable dispersing or wetting agents and suspending agents. The sterile injectable preparation can also be a sterile injectable solution or suspension in a nontoxic parenterally acceptable diluent or solvent, for example, as a solution in 1,3-butanediol. Among the acceptable vehicles and solvents that can be employed are water, Ringer's solution, and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid find use in the preparation of injectables.

The compounds according to the present invention can thus be formulated for parenteral administration (e.g. by injection, for example bolus injection or continuous infusion) and can be presented in unit dose form in ampoules, pre-filled syringes, small volume infusion or in multi-dose containers with an added preservative. The pharmaceutical compositions can take such forms as suspensions, solutions, or emulsions in oily or aqueous vehicles, and can contain formulatory agents such as suspending, stabilizing and/or dispersing agents. Alternatively, the active ingredient can be in powder form, obtained by aseptic isolation of sterile solid or by lyophilization from solution, for constitution with a suitable vehicle, e.g. sterile, pyrogen-free water, before use.

Aqueous solutions suitable for oral use can be prepared by dissolving the active component in water and adding suitable colorants, flavors, stabilizing and thickening agents, as desired.

Aqueous suspensions suitable for oral use can be made by dispersing the finely divided active component in water with viscous material, such as natural or synthetic gums, resins, methylcellulose, sodium carboxymethylcellulose, or other well known suspending agents.

Also included are solid form preparations which are intended to be converted, shortly before use, to liquid form preparations for oral administration. Such liquid forms include solutions, suspensions, and emulsions. These preparations can contain, in addition to the active component, colorants, flavors, stabilizers, buffers, artificial and natural sweeteners, dispersants, thickeners, solubilizing agents, and the like.

For topical administration to the epidermis the compounds according to the invention can be formulated as ointments, creams or lotions, or as a transdermal patch.

Ointments and creams can, for example, be formulated with an aqueous or oily base with the addition of suitable thickening and/or gelling agents. Lotions can be formulated with an aqueous or oily base and will in general also contain one or more emulsifying agents, stabilizing agents, dispersing agents, suspending agents, thickening agents, or coloring agents.

Formulations suitable for topical administration in the mouth include lozenges comprising active agent in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

Solutions or suspensions are applied directly to the nasal cavity by conventional means, for example with a dropper, pipette or spray. The formulations can be provided in single or multi-dose form. In the latter case of a dropper or pipette, this can be achieved by the patient administering an appropriate, predetermined volume of the solution or suspension. In the case of a spray, this can be achieved for example by means of a metering atomizing spray pump.

Administration to the respiratory tract can also be achieved by means of an aerosol formulation in which the active ingredient is provided in a pressurized pack with a suitable propellant. If the compounds disclosed herein or identified by methods of the invention or pharmaceutical compositions comprising them are administered as aerosols, for example as nasal aerosols or by inhalation, this can be carried out, for example, using a spray, a nebulizer, a pump nebulizer, an inhalation apparatus, a metered inhaler or a dry powder inhaler. Pharmaceutical forms for administration of the compounds disclosed herein or identified by methods of the invention as an aerosol can be prepared by processes well-known to the person skilled in the art. For their preparation, for example, solutions or dispersions of the compounds disclosed herein or identified by methods of the invention in water, water/alcohol mixtures or suitable saline solutions can be employed using customary additives, for example benzyl alcohol or other suitable preservatives, absorption enhancers for increasing the bioavailability, solubilizers, dispersants and others, and, if appropriate, customary propellants, for example include carbon dioxide, CFC's, such as, dichlorodifluoromethane, trichlorofluoromethane, or dichlorotetrafluoroethane; and the like. The aerosol can conveniently also contain a surfactant such as lecithin. The dose of drug can be controlled by provision of a metered valve.

In formulations intended for administration to the respiratory tract, including intranasal formulations, the compound will generally have a small particle size for example of the order of 10 microns or less. Such a particle size can be obtained by means known in the art, for example by micronization. When desired, formulations adapted to give sustained release of the active ingredient can be employed.

Alternatively the active ingredients can be provided in the form of a dry powder, for example, a powder mix of the compound in a suitable powder base such as lactose, starch, starch derivatives such as hydroxypropylmethyl cellulose and polyvinylpyrrolidone (PVP). Conveniently the powder carrier will form a gel in the nasal cavity. The powder composition can be presented in unit dose form for example in capsules or cartridges of, for example, gelatin, or blister packs from which the powder can be administered by means of an inhaler.

The pharmaceutical preparations can be in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are particularly useful compositions.

The invention also relates to a pharmaceutical composition comprising, consisting essentially of, or consisting of the glycemic stabilizing compound identified according to the methods of the invention disclosed herein.

For example, the invention relates to a pharmaceutical composition comprising, consisting essentially of, or consisting of the glycemic stabilizing compound, wherein said compound is identified by the method of: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention relates to a pharmaceutical composition comprising, consisting essentially of, or consisting of the glycemic stabilizing compound, wherein said compound is identified by the method of: a) contacting a candidate compound with a 5-HT₆ receptor, b) determining whether 5-HT₆ receptor functionality is increased, and c) determining whether a glycemic marker is directly modulated, wherein the modulation of the glycemic marker is first detected within less than 24 hours of the contacting of the candidate compound with the 5-HT₆ receptor, and wherein an increase in 5-HT₆ receptor functionality and direct modulation of a glycemic marker identifies the candidate compound as said glycemic stabilizing compound.

In a further embodiment, the invention relates to a pharmaceutical composition comprising, consisting essentially of, or consisting of the glycemic stabilizing compound, wherein said compound is identified by the method of: a) contacting a single dose of a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

In another embodiment, the invention relates to a pharmaceutical composition comprising, consisting essentially of, or consisting of the glycemic stabilizing compound, wherein said compound is identified by the method of: a) contacting a candidate compound with a 5-HT₆ receptor in an isolated cell, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

The pharmaceutical composition of the invention specifically excludes compounds already known in the art to be 5-HT₆ receptor agonists or partial agonists. For example, the pharmaceutical composition of the invention excludes BVT 5182, PRX-07034 and E-6837.

The invention thus further relates to pharmaceutical formulations comprising a compound identified by methods of the invention or a pharmaceutically acceptable salt or derivative thereof together with one or more pharmaceutically acceptable carriers thereof and/or prophylactic ingredients. The carrier(s) are “acceptable” in the sense of being compatible with the other ingredients of the formulation and not overly deleterious to the recipient thereof.

Pharmaceutical formulations include those suitable for oral, rectal, nasal, topical (including buccal and sub-lingual), vaginal or parenteral (including intramuscular, sub-cutaneous and intravenous) administration or in a form suitable for administration by inhalation or insufflation.

The compounds identified by the methods of the invention, together with a conventional adjuvant, carrier, or diluent, can thus be placed into the form of pharmaceutical formulations and unit dosages thereof, and in such form can be employed as solids, such as tablets or filled capsules, or liquids such as solutions, suspensions, emulsions, elixirs, gels or capsules filled with the same, all for oral use, in the form of suppositories for rectal administration; or in the form of sterile injectable solutions for parenteral (including subcutaneous) use. Such pharmaceutical compositions and unit dosage forms thereof can comprise conventional ingredients in conventional proportions, with or without additional active compounds or principles, and such unit dosage forms can contain any suitable effective amount of the active ingredient commensurate with the intended daily dosage range to be employed.

For oral administration, the pharmaceutical composition can be in the form of, for example, a tablet, capsule, suspension or liquid. The pharmaceutical composition can be made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins; with disintegrators such as corn starch, potato starch or sodium carboxymethyl-cellulose; and with lubricants such as talc or magnesium stearate. The active ingredient can also be administered by injection as a composition wherein, for example, saline, dextrose or water can be used as a suitable pharmaceutically acceptable carrier.

The dose when using the compounds identified by methods of the invention can vary within wide limits, and as is customary and is known to the physician, it is to be tailored to the individual conditions in each individual case. It depends, for example, on the nature and severity of the illness to be treated, on the condition of the patient, on the compound employed or on whether an acute or chronic disease state is treated or prophylaxis is conducted or on whether further active compounds are administered in addition to the compounds identified by methods of the invention. Representative doses of the present invention include, about 0.01 mg to about 1000 mg, about 0.01 to about 750 mg, about 0.01 to about 500 mg, 0.01 to about 250 mg, 0.01 mg to about 200 mg, about 0.01 mg to 150 mg, about 0.01 mg to about 100 mg, and about 0.01 mg to about 75 mg. Multiple doses can be administered during the day, especially when relatively large amounts are deemed to be needed, for example 2, 3 or 4, doses. If appropriate, depending on individual behavior and as appropriate from the patients physician or care-giver it can be necessary to deviate upward or downward from the daily dose.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. Typically, animal models include, but are not limited to, the rodent diabetes models as described in Example 18, infra (other animal models have been reported by Reed and Scribner in Diabetes, Obesity and Metabolism, 1:75-86 (1999)). In some circumstances, these extrapolations can merely be based on the weight of the animal model in comparison to another, such as a mammal, for example, a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated or prophylaxis is conducted or on whether further active compounds are administered in addition to the compounds disclosed herein or identified by methods of the invention and as part of a drug combination. The dosage regimen for treating a disease condition with the compounds and/or compositions of this invention is selected in accordance with a variety factors as cited above. Thus, the actual dosage regimen employed can vary widely and therefore can deviate from a preferred dosage regimen and one skilled in the art will recognize that dosage and dosage regimen outside these typical ranges can be tested and, where appropriate, can be used in the methods of this invention.

The desired dose can conveniently be presented in a single dose or as divided doses administered at appropriate intervals, for example, as two, three, four or more sub-doses per day. The sub-dose itself can be further divided, e.g., into a number of discrete loosely spaced administrations. The daily dose can be divided, especially when relatively large amounts are administered as deemed appropriate, into several, for example 2, 3 or 4, part administrations. If appropriate, depending on individual behavior, it can be necessary to deviate upward or downward from the daily dose indicated.

The compounds identified by methods of the invention can be administrated in a wide variety of oral and parenteral dosage forms. It will be obvious to those skilled in the art that the following dosage forms can comprise, as the active component, either a compound identified by methods of the invention or a pharmaceutically acceptable salt of a compound.

The invention also relates to a method for the manufacture of a medicament comprising a pharmaceutical composition comprising, consisting essentially of, or consisting of the glycemic stabilizing compound identified according to the methods of the invention, for use as a glycemic stabilizing compound. For example, the invention relates to a method for the manufacture of a medicament comprising a pharmaceutical composition comprising, consisting essentially of, or consisting of the glycemic stabilizing compound identified by the method comprising: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound.

The invention further relates to a method for the manufacture of a medicament comprising a pharmaceutical composition comprising, consisting essentially of, or consisting of the glycemic stabilizing compound identified according to the methods of the invention for use in the treatment of an insulin-related disorder. For example, the invention relates to a method for the manufacture of a medicament comprising a pharmaceutical composition comprising, consisting essentially of, or consisting of the glycemic stabilizing compound identified by the method comprising: a) contacting a candidate compound with a 5-HT₆ receptor, b) determining whether 5-HT₆ receptor functionality is increased, and c) determining whether a glycemic marker is directly modulated, wherein an increase in 5-HT₆ receptor functionality and direct modulation of a glycemic marker identifies the candidate compound as said glycemic stabilizing compound, for use in the treatment of an insulin-related disorder.

One aspect of the present invention pertains to a glycemic stabilizing compound, as identified by a method herein, for use in a method of treatment of the human or animal body by therapy.

Another aspect of the present invention pertains to a glycemic stabilizing compound, as identified by a method herein, for use in a method of treatment of an insulin related disorder, of the human or animal body by therapy. Another aspect of the present invention pertains to a method for the treatment of an insulin related disorder comprising administering to a subject suffering from said condition a therapeutically-effective amount of a glycemic stabilizing compound, as identified by a method herein.

One aspect of the present invention pertains to a method for the treatment of an insulin related disorder comprising administering to a subject suffering from said condition a therapeutically-effective amount of a glycemic stabilizing compound, as identified by a method herein, for example, in the form of a pharmaceutical composition. Another aspect of the present invention pertains to a glycemic stabilizing compound, as identified by a method herein, for use in a method of treatment of an insulin related disorder of the human or animal body by therapy.

The invention also relates to a method of identifying a candidate compound that results in an increase of blood glucose concentration, comprising: a) contacting a candidate compound with a 5-HT₆ receptor, b) determining whether 5-HT₆ receptor functionality is decreased, and c) determining whether a glycemic marker is directly modulated, wherein a decrease in 5-HT₆ receptor functionality and direct modulation of a glycemic marker identifies the candidate compound as a compound that results in an increase of blood glucose concentration. For example, a compound that decreases 5-HT₆ receptor functionality, such as a 5-HT₆ receptor antagonist, can result in a decrease in insulin secretion and an increase in blood glucose concentration. An increase in blood glucose can be desired, for example, in individuals with hypoglycemia.

The invention also relates to a method of identifying a candidate compound as an inhibitor of insulin secretion, comprising: a) contacting a candidate compound with a 5-HT₆ receptor, b) determining whether 5-HT₆ receptor functionality is decreased, and c) determining whether a glycemic marker is directly modulated, wherein a decrease in 5-HT₆ receptor functionality and direct modulation of a glycemic marker identifies the candidate compound as an inhibitor of insulin secretion. For example, a compound that decreases 5-HT₆ receptor functionality, such as a 5-HT₆ receptor antagonist, can result in a decrease in insulin secretion and an increase in blood glucose concentration. A decrease in insulin secretion can be desired, for example, in individuals with hypoglycemia.

Insulin-related disorders that could benefit from a decrease in insulin secretion include, for example, hypoglycemia, an insulin-secreting or insulin-dependent tumor, and aging.

Hypoglycemia is defined as abnormally low blood glucose. Hypoglycemia can result, for example, from excessive insulin or a poor diet. For example, hypoglycemia can occur when a person with diabetes has injected too much insulin, eaten too little food, or has exercised without extra food. Symptoms of hypoglycemia include, for example, a feeling of nervousness or weakness, headache, blurred vision, hunger, and excessive sweatiness.

Insulin-secreting tumors include, for example, insulinomas. An insulinoma is a tumor of the beta cells in areas of the pancreas called the islets of Langerhans. Although not usually cancerous, such tumors may cause the body to make extra insulin and may lead to a blood glucose level that is too low. In addition to insulin-secreting tumors, some tumors that do not secrete insulin can use insulin as a growth factor. While insulin may or may not be the sole growth factor used by the tumor, reduction in the amount of insulin in the body may reduce the growth of the tumor.

Aging is the physiological processes that occur in an organism as it gets older. Caloric restriction down-regulates insulin secretion and there is reason to suspect that these effects are key mediators of caloric restriction's favorable impact on longevity. Thus, strategies for down-regulating insulin can be useful to slow the process of aging and increase longevity.

In some embodiments, said glycemic stabilizing compound is a 5-HT6 receptor receptor inverse agonist or antagonist with an IC50 of less than 10 μM, of less than 1 μM, of less than 100 nM, or of less than 10 nM. In some embodiments, said glycemic stabilizing compound is an inverse agonist or antagonist with an IC50 of a value selected from the interval of 1 nM to 10 μM. In some embodiments, said glycemic stabilizing compound is an inverse agonist or antagonist with an IC50 of a value selected from the interval of 1 nM to 1 μM. In some embodiments, said glycemic stabilizing compound is an inverse agonist or antagonist with an IC50 of a value selected from the interval of 1 nM to 100 nM. In some embodiments, said glycemic stabilizing compound is an inverse agonist or antagonist with an IC50 of a value selected from the interval of 1 nM to 10 nM.

In certain embodiments, said IC50 is determined using an assay selected from the group consisting of: cyclase assay carried out using transfected cells expressing recombinant 5-HT6 receptor polypeptide; and melanophore assay carried out using transfected melanophores expressing recombinant 5-HT6 receptor polypeptide. In some embodiments, said glycemic stabilizing compound is an inverse agonist or antagonist with an IC50 of less than 10 μM, of less than 1 μM, of less than 100 nM, or of less than 10 nM in said assay. In some embodiments, said glycemic stabilizing compound is an inverse agonist or antagonist with an IC50 of less than 10 μM, of less than 9 μM, of less than 8 μM, of less than 7 μM, of less than 6 μM, of less than 5 μM, of less than 4 μM, of less than 3 μM, of less than 2 μM, of less than 1 μM, of less than 900 nM, of less than 800 nM, of less than 700 nM, of less than 600 nM, of less than 500 nM, of less than 400 nM, of less than 300 nM, of less than 200 nM, of less than 100 nM, of less than 90 nM, of less than 80 nM, of less than 70 nM, of less than 60 nM, of less than 50 nM, of less than 40 nM, of less than 30 nM, of less than 20 nM, of less than 10 nM in said assay. In some embodiments, said glycemic stabilizing compound is an inverse agonist or antagonist with an IC50 in said assay of a value selected from the interval of 1 nM to 10 μM. In some embodiments, said glycemic stabilizing compound is an inverse agonist or antagonist with an IC50 in said assay of a value selected from the interval of 1 nM to 1 μM. In some embodiments, said glycemic stabilizing compound is an inverse agonist or antagonist with an IC50 in said assay of a value selected from the interval of 1 nM to 100 nM. In some embodiments, said glycemic stabilizing compound is an inverse agonist or antagonist with an IC50 in said assay of a value selected from the interval of 1 nM to 10 nM. In some embodiments, said glycemic stabilizing compound is selective for the 5-HT6 receptor GPCR.

One object of the invention relates to a method of (a) performing a method of the invention to identify a glycemic stabilizing compound and (b) optionally, determining the structure of the compound, and (c) providing the compound or the name or structure of the compound. In addition, the invention relates to a method of (a) performing a method of the invention to identify a glycemic stabilizing compound and (b) optionally, determining the structure of the compound, (c) optionally, providing the name or structure of the compound, and (d) producing or synthesizing the compound. The invention further relates to a process for modulating the functionality of a GPCR comprising performing a method of the invention to identify a glycemic stabilizing compound and then contacting the GPCR with the glycemic stabilizing compound or administering the glycemic stabilizing compound to an individual under conditions sufficient to modulate the functionality of the GPCR.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. In addition, all subcombinations of uses and medical indications described herein are also specifically embraced by the present invention just as if every subcombination of uses and medical indications was individually and explicitly recited herein.

Applicants reserve the right to exclude any one or more candidate compounds from any of the embodiments of the invention. Applicants also reserve the right to exclude any insulin-related disorder, or any condition related to an elevated blood glucose concentration from any of the embodiments of the invention.

Other uses of the disclosed receptors and methods will become apparent to those in the art based upon, inter alia, a review of this patent document.

The following examples are given to illustrate the invention and are not intended to be inclusive in any manner:

### EXAMPLES

The examples are provided to further define the invention without, however, limiting the invention to the specifics of these examples.

### Example 1

### RT-PCR Analysis of 5-HT₆ Receptor Expression in Mouse Tissues and Transformed Endocrine Cells

In this example, the expression level of mouse 5-HT₆ receptor was determined in several mouse tissues and transformed endocrine cells. As shown in FIG. 1, expression of the mouse 5-HT₆ receptor was detected in the brain, two samples of GLUTag cells, Min6, Nit-1 and alphaTC1-9 cells.

Total RNA was isolated from mouse tissues collected from C57bl/6 male mice and mouse endocrine cell lines using RNAzol B reagent following reagent provider's recommended protocol. RNA samples were further treated with Ambion Turbo DNAse kit (Ambion Cat # 1907) to remove possible genomic DNA contamination. Genomic DNA-free RNA preparation from above was confirmed by PCR amplification of β-actin using RT-minus control (FIG. 1, bottom panel). For RT-PCR analysis of 5-HT₆ receptor expression in the indicated mouse tissues and cell lines, genomic DNA-free RNA were converted to cDNA using a cDNA synthesis kit purchased from BioRad (iScript cDNA synthesis kit—BioRad cat #170-8891). The PCR amplification was done using mouse 5-HT₆ receptor-specific oligonucleotide primers (forward primer: 5′-TGCTAGCTCGCGGCCTCTGTCTGC-3′ SEQ ID NO.3; reverse primer: 5′-CATCCCTGGGCGTGGTGTCCTG-3′ SEQ ID NO.4), with the following amplification sequence: [94° C. for 4 minutes; 36×(94° C. for 1 minute, 65° C. for 30 seconds, 72° C. for 1 minute), and 72° C. for 7 minutes]. Amplified products were separated via agarose gel electrophoresis and visualized under UV light after ethidium bromide staining.

### Example 2

RT-PCR Analysis of 5-HT₆ Receptor Expression in ob/ob Mouse Tissue

In this example, the expression level of mouse 5-HT₆ receptor was determined in several mouse tissues derived from three different ob/ob mutant obese mice. As shown in FIG. 2, expression of the mouse 5-HT₆ receptor was detected in the brain, duodenum, small intestine and colon of these mice.

Ob mice (approximately 10 weeks old) were euthanized and perfused in fixatives of 4% paraformaldyhade/PBS solution. Tissues (brain, pancreas, liver, duodenum, jejunum/ileum, and colon) were collected and snap-frozen in liquid nitrogen. Total RNA was isolated using RNAzol B reagent following reagent provider's recommended protocol. RNA samples were further treated with Ambion Turbo DNAse kit (Ambion Cat # 1907) to remove possible genomic DNA contamination. Genomic DNA-free RNA preparation from above was confirmed by PCR amplification of β-actin using RT-minus control. For RT-PCR analysis of 5-HT₆ receptor, genomic DNA-free RNA were converted to cDNA using a cDNA synthesis kit purchased from BioRad (iScript cDNA synthesis kit—BioRad cat #170-8891). The PCR amplification was done using mouse 5-HT6-specific oligonucleotide primers (forward primer: 5′-GCCGCCAATTCGCTGCTGAT-3′ SEQ ID NO.5; reverse primer: 5′-ACGCCGGACGCCACGAGGACATA-3′ SEQ ID NO.6), with the following amplification sequence: [94° C. for 4 minutes; 30×(94° C. for 1 minute, 63° C. for 30 seconds, 72° C. for 1 minute), and 72° C. for 7 minutes] (result shown in Top panel), or a PCR amplification sequence [94° C. for 4 minutes; 36×(94° C. for 1 minute, 63° C. for 30 seconds, 72° C. for 1 minute), and 72° C. for 7 minutes] (result shown in middle panel). GAPDH was used as cDNA loading control. Amplified products were separated via agarose gel electrophoresis and visualized under UV light after ethidium bromide staining.

### Example 3

RT-PCR Analysis of 5-HT₆ Receptor Expression in the Pancreas of ob/ob and C57Bl/6 Mice

In this example, the expression level of mouse 5-HT₆ receptor was determined in the pancreas of three different ob/ob mutant obese mice and two different wild-type C57Bl/6 mice. As shown in FIG. 3, the mouse 5-HT₆ receptor was not detected in the pancreas of ob/ob or C57Bl/6 mice, while it was expressed in the brain and genomic DNA positive controls.

Three ob/ob mice (approximately 10 weeks old) and two C57Bl/6 mice (approximately 10 weeks old) were euthanized and perfused in fixatives of 4% paraformaldyhade/PBS solution. Pancreas was collected and snap-frozen in liquid nitrogen. Total RNA was isolated using RNAzol B reagent following reagent provider's recommended protocol. RNA samples were further treated with Ambion Turbo DNAse kit (Ambion Cat # 1907) to remove possible genomic DNA contamination. Genomic DNA-free RNA preparation from above was confirmed by PCR amplification of β-actin using RT-minus control. For RT-PCR analysis of 5-HT₆ receptor, genomic DNA-free RNA were converted to cDNA using a cDNA synthesis kit purchased from BioRad (iScript cDNA synthesis kit—BioRad cat #170-8891). The PCR amplification was done using mouse 5-HT₆ receptor-specific oligonucleotide primers (forward primer: 5′-GCCGCCAATTCGCTGCTGAT-3′ SEQ ID NO. 5; reverse primer: 5′-ACGCCGGACGCCACGAGGACATA-3′ SEQ ID NO.6), with the following amplification sequence: [94° C. for 4 minutes; 30×(94° C. for 1 minute, 63° C. for 30 seconds, 72° C. for 1 minute), and 72° C. for 7 minutes] (result shown in Top panel), or a PCR amplification sequence [94° C. for 4 minutes; 36×(94° C. for 1 minute, 63° C. for 30 seconds, 72° C. for 1 minute), and 72° C. for 7 minutes] (result shown in middle panel). GAPDH was used as cDNA loading control. Amplified products were separated via agarose gel electrophoresis and visualized under UV light after ethidium bromide staining.

### Example 4

### Cyclase Assays for Mouse 5-HT₆ Receptor Agonist Compound A

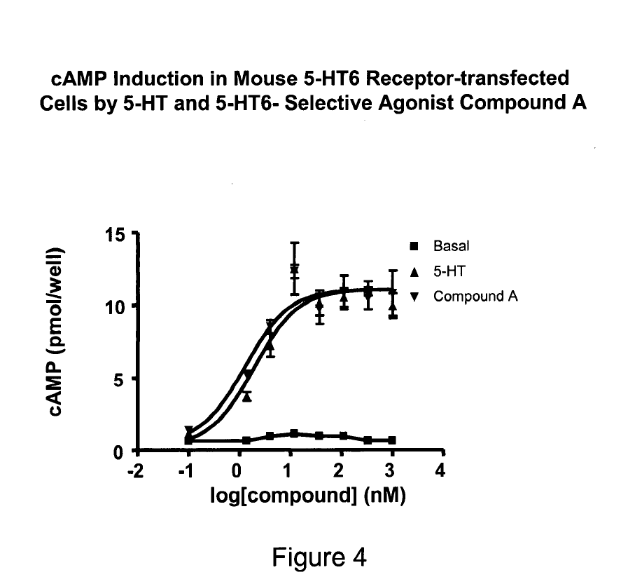

In this example, a mouse 5-HT₆ receptor agonist, for example, Compound A (5-Chloro-2-methyl-3-(1,2,3,6-tetrahydro-pyridin-4-yl)-1H-indole) was tested in a cyclase assay. As shown in FIG. 4, Compound A stimulates cAMP production in a cyclase assay.

Cyclic AMP measurements were done with a Flash Plate™ Adenylyl Cyclase kit (New England Nuclear) according to the supplier's protocol. Briefly, HEK293 cells were transfected with either empty vector DNA (Basal) or mouse 5-HT₆ receptor expression plasmid DNA using Lipofectamine (Invitrogen, Carlsbad, Calif.). After 24 hours, transfected cells were harvested in GIBCO cell dissociation buffer (Cat #13151-014) and re-suspended in Assay Buffer (50% 1×PBS/50% Stimulation Buffer). Compounds (5-HT or EMD 386088) of various doses were incubated with 10⁵ cells/well for 60 minutes at room temperature. After further 2 hour incubation with tracer in detection buffer, plates were counted in a Wallac MicroBeta scintillation counter. Values of cAMP/well were extrapolated from a standard cAMP curve which was included on each assay plate, and plotted using Prism graphing program.

### Example 5

A 5-HT₆ Receptor Agonist Directly Improves oGTT in C57/Bl6 Mice

In this example, the effect of mouse 5-HT₆ receptor agonist Compound A on glucose excursion in an oral glucose tolerance test (oGTT) was determined. As shown in FIG. 5, top panels, Compound A improved glucose excursion in a dose-dependent fashion in C57/Bl6 mice.

Oral Glucose Tolerance: overnight fasted C57bl/6 male mice (n=6 mice per treatment) were treated via oral gavage with vehicle (20% HPCD), or dosed with Compound A at 1 mpk, 3.3 mpk, or 10 mpk (milligram compound per kilogram of body weight). Thirty minutes later, a glucose bolus (3 gram/kg) was then delivered per orally. Plasma glucose levels were determined at indicated time points over a two hour period using blood (˜5 μl) collected from tail nick and a glucose meter. Glycemic excursion curve was graphed based on data from 6 mice and given in mean values +/−SEM (FIG. 5 upper left panel).

The same test can be performed using i.p. administration of dextrose as shown in FIG. 5, upper right panel.

### Example 6

### A 5-HT₆ Receptor Agonist Increases Insulin Release in Response to a Glucose Bolus

In this example, the effect of mouse 5-HT₆ receptor agonist Compound A on insulin release in mice was determined. As shown in FIG. 5, bottom panel, Compound A increased insulin release in compared to vehicle at 5, 10 and 20 minutes after a glucose bolus.

C57bl/6 male mice (8 weeks of age) were fasted for 18 hours, and randomly assigned into eight groups with n=6 for each group. Mice were administered per orally with vehicle (20% HPCD) or with 10 mpk EMD 386088. After 30 minutes, a glucose bolus of 3 g/kg was administered orally. Blood was collected in heparinated blood collection tubes at 0 minute (no glucose bolus), 5 minutes, 10 minutes, and 20 minutes after glucose bolus. Plasma samples were obtained via centrifugation at 500×g for 20 minutes and assayed for insulin using an Ultra Sensitive Insulin ELISA kit (Crystal Chem. Inc., Downers Grove, Ill.).

### Example 7

Robust Insulin Release in Mice Treated with a Combination of a DPP-IV Inhibitor and a 5-HT₆ Receptor Agonist

In this example, the effect of mouse 5-HT₆ receptor agonist Compound A in combination with a DPP-IV inhibitor on insulin release in mice was determined. As shown in FIG. 6, the DPP-IV inhibitor by itself increased insulin release in mice compared to vehicle at 3 minutes after a glucose bolus. The combination of DPP-IV inhibitor resulted in a greater release of insulin compared to DPP-IV inhibitor alone at 3 minutes after a glucose bolus.

C57blk/6 male mice (8 weeks of age) were fasted for 18 hours, and randomly assigned into 6 groups with n=6 for each group. Mice were administered per orally with vehicle (20% HPCD), DPP-IV inhibitor (3 mpk), or a combination of DPP-IV inhibitor (3 mpk) and Compound A (10 mpk), as indicated. Thirty minutes after treatment, a glucose bolus at 3 g/kg was delivered per orally, and plasma was collected at 0 minute, (no glucose bolus), and 3 minutes after glucose bolus. Plasma insulin levels were determined by using an Ultra Sensitive Insulin ELISA kit (Crystal Chem. Inc., Downers Grove, Ill.).

### Example 8

A 5-HT₆ Receptor Agonist does not Cause Hypoglycemia

In this example, the effect of 5-HT₆ receptor agonist Compound A on plasma glucose levels in mice was determined. As shown in FIG. 7, 5-HT₆ receptor agonist Compound A did not cause hypoglycemia compared to the control.

Overnight fasted C57bl/6 male mice (n=6 mice per treatment) were treated via oral gavage with vehicle (20% HPCD), or dosed with Compound A at 10 mpk or 30 mpk (milligram compound per kilogram of body weight). Plasma glucose levels were determined at 0 minute (right before dosing), and 30, 60, 90, and 120 minutes post-dosing using blood (˜5 μl) collected from tail nick and a glucose meter. Glycemic changes were graphed based on data from 6 mice and given in mean values +/−SEM.

### Example 9

5-HT₆ Receptor Agonist Compound A Improves oGTT in Diabetic db/db Mice

In this example, the effect of mouse 5-HT₆ receptor agonist Compound A on oGTT in db/db mutant diabetic mice was tested. As shown in FIG. 8, Compound A resulted in a dose dependent decrease in plasma glucose concentration over time compared to the control treatment.

Diabetic db/db mice (˜8 weeks old) were fasted for 4 hours (n=6 mice per treatment) and then treated via oral gavage with vehicle (20% HPCD), or dosed with Compound A at 3.3 mpk, 10 mpk, or 30 mpk (milligram compound per kilogram of body weight). Thirty minutes later, a glucose bolus (2 gram/kg) was then delivered per orally. Plasma glucose levels were determined at indicated time points over a two hour period using blood (˜5 μl) collected from tail nick and a glucose meter. Glycemic excursion curve was graphed based on data from 6 mice and given in mean values +/−SEM.

### Example 10

### Melanophore Technology

Melanophores are skin cells found in lower vertebrates. They contain pigmented organelles termed melanosomes. Melanophores are able to redistribute these melanosomes along a microtubule network upon G-protein coupled receptor (GPCR) activation. The result of this pigment movement is an apparent lightening or darkening of the cells. In melanophores, the decreased levels of intracellular cAMP that result from activation of a Gi-coupled receptor cause melanosomes to migrate to the center of the cell, resulting in a dramatic lightening in color. If cAMP levels are then raised, following activation of a Gs-coupled receptor, the melanosomes are re-dispersed and the cells appear dark again. The increased levels of diacylglycerol that result from activation of Gq-coupled receptors can also induce this re-dispersion. In addition, the technology is also suited to the study of certain receptor tyrosine kinases. The response of the melanophores takes place within minutes of receptor activation and results in a simple, robust color change. The response can be easily detected using a conventional absorbance microplate reader or a modest video imaging system. Unlike other skin cells, the melanophores derive from the neural crest and appear to express a full complement of signaling proteins. In particular, the cells express an extremely wide range of G-proteins and so are able to functionally express almost all GPCRs.

Melanophores can be utilized to identify compounds, including natural ligands, which bind to and/or activate GPCRs. This method can be conducted by introducing test cells of a pigment cell line capable of dispersing or aggregating their pigment in response to a specific stimulus and expressing an exogenous clone coding for the GPCR. An initial state of pigment disposition can be set using, for example, using melatonin, MSH or light. The test cells are then contacted with chemical compounds, and it is determined whether the pigment disposition in the cells changed from the initial state of pigment disposition. Dispersion of pigments cells due to the candidate compound, including but not limited to a ligand, coupling to the GPCR will appear dark on a petri dish, while aggregation of pigments cells will appear light.

Materials and methods are followed according to the disclosure of U.S. Pat. No. 5,462,856 and U.S. Pat. No. 6,051,386. These patent disclosures are hereby incorporated by reference in their entirety.

Melanophores are transfected by electroporation with a plasmid which contains the coding sequence of a 5HT-6 receptor. The cells are plated in 96-well plates. 48 hours post-transfection, half of the cells on each plate are treated with 10 nM melatonin. Melatonin activates an endogenous Gi-coupled receptor in the melanophores and causes them to aggregate their pigment. The remaining half of the cells are transferred to serum-free medium 0.7×L-15 (Gibco). After one hour, the cells in serum-free media remain in a pigment-dispersed state while the melatonin-treated cells are in a pigment-aggregated state. At this point, the cells are treated with different compounds from a proprietary compound library containing hundreds of thousands of organic small molecule compounds. If 5-HT₆ receptor bound to the compound, the melanophores would be expected to undergo a color change in response to the compound. Since the receptor can couple to Gs, the pigment-dispersed cells should undergo a dose-dependent pigment dispersion.

### Example 11

### Assays for Determination of GPCR Activation

A variety of approaches are available for assessment of activation of human GPCRs. The following are illustrative; those of ordinary skill in the art are credited with the ability to determine those techniques that are preferentially beneficial for the needs of the artisan.

1. Membrane Binding Assays: [³⁵S]GTPγS Assay

When a G protein-coupled receptor is in its active state, either as a result of ligand binding or constitutive activation, the receptor couples to a G protein and stimulates the release of GDP and subsequent binding of GTP to the G protein. The alpha subunit of the G protein-receptor complex acts as a GTPase and slowly hydrolyzes the GTP to GDP, at which point the receptor normally is deactivated. Activated receptors continue to exchange GDP for GTP. The non-hydrolyzable GTP analog, [³⁵S]GTPγS, can be utilized to demonstrate enhanced binding of [³⁵S]GTPγS to membranes expressing activated receptors. The advantage of using [³⁵S]GTPγS binding to measure activation is that: (a) it is generically applicable to all G protein-coupled receptors; (b) it is proximal at the membrane surface making it less likely to pick-up molecules which affect the intracellular cascade.

The assay utilizes the ability of G protein coupled receptors to stimulate [³⁵S]GTPγS binding to membranes expressing the relevant receptors. The assay can, therefore, be used in the direct identification method to screen candidate compounds to endogenous GPCRs and non-endogenous, constitutively activated GPCRs. The assay is generic and has application to drug discovery at all G protein-coupled receptors.

The [³⁵S]GTPγS assay is performed using a cell which expresses the receptor of interest. Generally, the cells are harvested and a pellet containing the membrane fraction which contains the receptor of interest is frozen. To perform the assay, the membrane pellet is thawed (if frozen) on ice and homogenized briefly until in suspension using a polytron mixer (POLYTRON PT3100, probe PT-DA 3007/2 at setting of 7000 rpm). The membrane protein concentration is determined using a Bradford assay. The membrane protein is diluted to a protein concentration of 0.20 mg/ml in Binding Buffer (20 mM HEPES, pH 7.4, 100 mM NaCl, 10 mM MgCl₂) for a final assay concentration of 5 μg/well. Compound plates to be screened are thawed (daughter plates with 5 μL compound @ 2 mM in 100% DMSO). The 2 mM compounds are diluted 1:50 with 245 μL GDP buffer (binding buffer plus GDP, Sigma-Aldrich Catalog #87127, ranging from 0.4 to 40 μM, made fresh before assay) to 40 μM in 2% DMSO. Compounds in GDP buffer (25 μl) are added per well of Scintistrip plate (Wallac Catalog #1450-501) and then 50 μl of membrane preparation (0.4 mg protein/ml) is added to each well. The plates are then covered with foil and incubated for 5-10 minutes at room temperature. Subsequently, 25 μl of diluted [³⁵S]GTPγS (Amersham Biosciences Catalog #SJ1320, ˜2000 Ci/mmol—this is made by adding 5 μl [³⁵S]GTPγS stock into 10 ml binding buffer) is added to the wells. The plates are covered with foil and incubated on the shaker (Lab-Line model #1314, at setting of 4) for 60 minutes at room temperature. The assay is stopped by sealing the plates with plate covers and spinning the plates at 4000 rpm for 15 minutes at 22° C. The supernatant of each well is aspirated using an 8-channel manifold and the plate is read in a Wallac Microbeta counter 1450 set up to detect ³⁵S.

2. Adenylyl Cyclase

A Flash Plate™ Adenylyl Cyclase kit (New England Nuclear; Cat. No. SMP004A) designed for cell-based assays can be modified for use with crude plasma membranes. The Flash Plate wells can contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express a receptor.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS are added to each plate. Cells are pipetted off the plate and the cell suspension is collected into a 50 ml conical centrifuge tube. Cells are then centrifuged at room temperature at 1,100 rpm for 5 minutes. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer is prepared fresh for screening and contains 50 μl of Stimulation Buffer, 3 μl of candidate compound (12 μM final assay concentration) and 50 μl cells. Assay Buffer is stored on ice until utilized. The assay, preferably carried out, for example, in a 96-well plate, is initiated by addition of 50 μl of cAMP standards to appropriate wells followed by addition of 50 μl of PBSA to wells H11 and H12. 50 μl of Stimulation Buffer is added to all wells. DMSO (or selected candidate compounds) is added to appropriate wells using a pin tool capable of dispensing 3 μl of compound solution, with a final assay concentration of 12 μM candidate compound and 100 μl total assay volume. The cells are then added to the wells and incubated for 60 minutes at room temperature. 100 μl of Detection Mix containing tracer cAMP is then added to the wells. Plates are then incubated additional 2 hours followed by counting in a Wallac MicroBeta scintillation counter. Values of cAMP/well are then extrapolated from a standard cAMP curve which is contained within each assay plate.

3. Cell-Based cAMP for Gi Coupled Target GPCRs

TSHR is a Gs coupled GPCR that causes the accumulation of cAMP upon activation. TSHR can be constitutively activated by mutating amino acid residue 623 (i.e., changing an alanine residue to an isoleucine residue). A Gi coupled receptor is expected to inhibit adenylyl cyclase, and, therefore, decrease the level of cAMP production, which can make assessment of cAMP levels challenging. An effective technique for measuring the decrease in production of cAMP as an indication of activation of a Gi coupled receptor can be accomplished by co-transfecting, non-endogenous, constitutively activated TSHR (TSHR-A623I) (or an endogenous, constitutively active Gs coupled receptor) as a “signal enhancer” with a Gi linked target GPCR to establish a baseline level of cAMP. Upon creating an endogenous or non-endogenous version of the Gi coupled receptor, the target GPCR is then co-transfected with the signal enhancer, and it is this material that can be used for screening. In some embodiments, this approach is preferably used in the direct identification of candidate compounds against Gi coupled receptors. It is noted that for a Gi coupled GPCR, when this approach is used, an inverse agonist of the target GPCR will increase the cAMP signal and an agonist will decrease the cAMP signal.

On day one, 2×10⁴ 293 cells/well are plated out. On day two, two reaction tubes are prepared (the proportions to follow for each tube are per plate): tube A is prepared by mixing 2 μg DNA of each receptor transfected into the mammalian cells, for a total of 4 μg DNA (e.g., pCMV vector; pCMV vector with mutated THSR (TSHR-A623I); TSHR-A623I and GPCR, etc.) in 1.2 ml serum free DMEM (Irvine Scientific, Irvine, Calif.); tube B is prepared by mixing 120 μl lipofectamine (Gibco BRL) in 1.2 ml serum free DMEM. Tubes A and B are then admixed by inversions (several times), followed by incubation at room temperature for 30-45 minutes. The admixture is referred to as the “transfection mixture”. Plated 293 cells are washed with 1×PBS, followed by addition of 10 ml serum free DMEM. 2.4 ml of the transfection mixture is then added to the cells, followed by incubation for 4 hours at 37° C./5% CO₂. The transfection mixture is then removed by aspiration, followed by the addition of 25 ml of DMEM/10% Fetal Bovine Serum. Cells are then incubated at 37° C./5% CO₂. After 24 hours incubation, cells are harvested and utilized for analysis.

A Flash Plate™ Adenylyl Cyclase kit (New England Nuclear; Cat. No. SMP004A) is designed for cell-based assays, but can be modified for use with crude plasma membranes depending on the need of the skilled artisan. The Flash Plate wells contain a scintillant coating which also contains a specific antibody recognizing cAMP. The cAMP generated in the wells can be quantitated by a direct competition for binding of radioactive cAMP tracer to the cAMP antibody. The following serves as a brief protocol for the measurement of changes in cAMP levels in whole cells that express a receptor of interest.

Transfected cells are harvested approximately twenty four hours after transient transfection. Media is carefully aspirated off and discarded. 10 ml of PBS is gently added to each dish of cells followed by careful aspiration. 1 ml of Sigma cell dissociation buffer and 3 ml of PBS is added to each plate. Cells are pipetted off the plate and the cell suspension is collected into a 50 ml conical centrifuge tube. Cells are then centrifuged at room temperature at 1,100 rpm for 5 minutes. The cell pellet is carefully re-suspended into an appropriate volume of PBS (about 3 ml/plate). The cells are then counted using a hemocytometer and additional PBS is added to give the appropriate number of cells (with a final volume of about 50 μl/well).

cAMP standards and Detection Buffer (comprising 1 μCi of tracer [¹²⁵I] cAMP (50 μl) to 11 ml Detection Buffer) is prepared and maintained in accordance with the manufacturer's instructions. Assay Buffer should be prepared fresh for screening and contain 50 μl of Stimulation Buffer, 3 μl of candidate compound (12 μM final assay concentration) and 50 μl cells. Assay Buffer can be stored on ice until utilized. The assay can be initiated by addition of 50 μl of cAMP standards to appropriate wells followed by addition of 50 μl of PBSA to wells H-11 and H12. Fifty μl of Stimulation Buffer is added to all wells. Selected compounds (e.g., TSH) are added to appropriate wells using a pin tool capable of dispensing 3 μl of compound solution, with a final assay concentration of 12 μM candidate compound and 100 μl total assay volume. The cells are then added to the wells and incubated for 60 minutes at room temperature. 100 μl of Detection Mix containing tracer cAMP is then added to the wells. Plates are then incubated additional 2 hours followed by counting in a Wallac MicroBeta scintillation counter. Values of cAMP/well are extrapolated from a standard cAMP curve which is contained within each assay plate.

4. Reporter-Based Assays

a. CRE-LUC Reporter Assay (Gs-Associated Receptors)

293 or 293T cells are plated-out on 96 well plates at a density of 2×10⁴ cells per well and are transfected using Lipofectamine Reagent (BRL) the following day according to manufacturer instructions. A DNA/lipid mixture is prepared for each E-well transfection as follows: 260 ng of plasmid DNA in 100 μl of DMEM is gently mixed with 2 μl of lipid in 100 μl of DMEM (the 260 ng of plasmid DNA consists of 200 ng of a 8×CRE-Luc reporter plasmid, 50 ng of pCMV comprising endogenous receptor or non-endogenous receptor or pCMV alone, and 50 ng of a GPRS expression plasmid (GPRS in pcDNA3 (Invitrogen)). The 8×CRE-Luc reporter plasmid is prepared as follows: vector SRIF-β-gal is obtained by cloning the rat somatostatin promoter (−71/+51) at BglV-HindIII site in the pβgal-Basic Vector (Clontech). Eight (8) copies of cAMP response element are obtained by PCR from an adenovirus template AdpCF126CCRE8 (see, Suzuki et al., Hum Gene Ther 7:1883-1893 (1996); the disclosure of which is hereby incorporated by reference in its entirety) and cloned into the SRIF-β-gal vector at the Kpn-BglV site, resulting in the 8×CRE-β-gal reporter vector. The 8×CRE-Luc reporter plasmid is generated by replacing the beta-galactosidase gene in the 8×CRE-β-gal reporter vector with the luciferase gene obtained from the pGL3-basic vector (Promega) at the HindIII-BamHI site. Following 30 minutes incubation at room temperature, the DNA/lipid mixture is diluted with 400 μl of DMEM and 100 μl of the diluted mixture is added to each well. 100 μl of DMEM with 10% FCS are added to each well after a four hour incubation in a cell culture incubator. The following day the transfected cells are changed with 200 μl/well of DMEM with 10% FCS. Eight (8) hours later, the wells are changed to 100 μl/well of DMEM without phenol red, after one wash with PBS. Luciferase activity is measured the next day using the LucLite™ reporter gene assay kit (Packard) following manufacturer instructions and read on a 1450 MicroBeta™ scintillation and luminescence counter (Wallac).

b. AP1 Reporter Assay (Gq-Associated Receptors)

A method to detect Gq stimulation depends on the known property of Gq-dependent phospholipase C to cause the activation of genes containing AP1 elements in their promoter. A Pathdetect™ AP-1 cis-Reporting System (Stratagene, Catalogue No. 219073) can be utilized following the protocol set forth above with respect to the CREB reporter assay, except that the components of the calcium phosphate precipitate are 410 ng pAP1-Luc, 80 ng pCMV-receptor expression plasmid, and 20 ng CMV-SEAP.

c. SRF-LUC Reporter Assay (Gq-Associated Receptors)

One method to detect Gq stimulation depends on the known property of Gq-dependent phospholipase C to cause the activation of genes containing serum response factors in their promoter. A Pathdetect™ SRF-Luc-Reporting System (Stratagene) can be utilized to assay for Gq coupled activity in, for example, COS7 cells. Cells are transfected with the plasmid components of the system and the indicated expression plasmid encoding endogenous or non-endogenous GPCR using a Mammalian Transfection™ Kit (Stratagene, Catalogue #200285) according to the manufacturer's instructions. Briefly, 410 ng SRF-Luc, 80 ng pCMV-receptor expression plasmid and 20 ng CMV-SEAP (secreted alkaline phosphatase expression plasmid; alkaline phosphatase activity is measured in the media of transfected cells to control for variations in transfection efficiency between samples) are combined in a calcium phosphate precipitate as per the manufacturer's instructions. Half of the precipitate is equally distributed over 3 wells in a 96-well plate and kept on the cells in a serum free media for 24 hours. The last 5 hours the cells are incubated with, for example, 1 μM, candidate compound. Cells are then lysed and assayed for luciferase activity using a Luclite™ Kit (Packard, Cat. No. 6016911) and “Trilux 1450 Microbeta” liquid scintillation and luminescence counter (Wallac) as per the manufacturer's instructions. The data can be analyzed using GraphPad Prism™ 2.0a (GraphPad Software Inc.).

d. Intracellular IP3 Accumulation Assay (Gq-Associated Receptors)

On day 1, cells comprising the receptor of interest (endogenous or non-endogenous) can be plated onto 24 well plates, usually 1×10⁵ cells/well (although his number can be optimized). On day 2 cells can be transfected by first mixing 0.25 μg DNA in 50 μl serum free DMEM/well and 2 μl lipofectamine in 50 μl serum free DMEM/well. The solutions are gently mixed and incubated for 15-30 minutes at room temperature. Cells are washed with 0.5 ml PBS and 400 μl of serum free media is mixed with the transfection media and added to the cells. The cells are then incubated for 3-4 hours at 37° C./5% CO₂ and then the transfection media is removed and replaced with 1 ml/well of regular growth media. On day 3 the cells are labeled with ³H-myo-inositol. Briefly, the media is removed and the cells are washed with 0.5 ml PBS. Then 0.5 ml inositol-free/serum free media (GIBCO BRL) is added/well with 0.25 μCi of ³H-myo-inositol/well and the cells are incubated for 16-18 hours overnight at 37° C./5% CO₂. On Day 4 the cells are washed with 0.5 ml PBS and 0.45 ml of assay medium is added containing inositol-free/serum free media, 10 μM pargyline, 10 mM lithium chloride or 0.4 ml of assay medium and 50 μl of 10× ketanserin (ket) to final concentration of 10 μM, if using a control construct containing a serotonin receptor. The cells are then incubated for 30 minutes at 37° C. The cells are then washed with 0.5 ml PBS and 200 μl of fresh/ice cold stop solution (1M KOH; 18 mM Na-borate; 3.8 mM EDTA) is added/well. The solution is kept on ice for 5-10 minutes or until cells were lysed and then neutralized by 200 μl of fresh/ice cold neutralization sol. (7.5% HCL). The lysate is then transferred into 1.5 ml eppendorf tubes and 1 ml of chloroform/methanol (1:2) is added/tube. The solution is vortexed for 15 seconds and the upper phase is applied to a Biorad AG1-X8™ anion exchange resin (100-200 mesh). Firstly, the resin is washed with water at 1:1.25 W/V and 0.9 ml of upper phase is loaded onto the column. The column is washed with 10 mls of 5 mM myo-inositol and 10 ml of 5 mM Na-borate/60 mM Na-formate. The inositol tris phosphates are eluted into scintillation vials containing 10 ml of scintillation cocktail with 2 ml of 0.1 M formic acid/1 M ammonium formate. The columns are regenerated by washing with 10 ml of 0.1 M formic acid/3M ammonium formate and rinsed twice with dd H₂O and stored at 4° C. in water.

### Example 12

### Fusion Protein Preparation

a. GPCR:Gs Fusion Constuct

The design of the GPCR-G protein fusion construct can be accomplished as follows: both the 5′ and 3′ ends of the rat G protein Gsα (long form; Itoh, H. et al., Proc. Natl. Acad. Sci. 83:3776 (1986)) are engineered to include a HindIII sequence thereon. Following confirmation of the correct sequence (including the flanking HindIII sequences), the entire sequence is shuttled into pcDNA3.1(−) (Invitrogen, cat. no. V795-20) by subcloning using the HindIII restriction site of that vector. The correct orientation for the Gsα sequence is determined after subcloning into pcDNA3.1(−). The modified pcDNA3.1(−) containing the rat Gsα gene at HindIII sequence is then verified; this vector is now available as a “universal” Gsα protein vector. The pcDNA3.1(−) vector contains a variety of well-known restriction sites upstream of the HindIII site, thus beneficially providing the ability to insert, upstream of the Gs protein, the coding sequence of a receptor of interest. This same approach can be utilized to create other “universal” G protein vectors, and, of course, other commercially available or proprietary vectors known to the artisan can be utilized—the important criteria is that the sequence for the GPCR be upstream and in-frame with that of the G protein.

b. Gq(6 Amino Acid Deletion)/Gi Fusion Construct

The design of a Gq(del)/Gi fusion construct can be accomplished as follows: the N-terminal six (6) amino acids (amino acids 2 through 7, having the sequence of TLESIM (SEQ ID NO:7)) of Gαq-subunit is deleted and the C-terminal five (5) amino acids having the sequence EYNLV (SEQ ID NO:8) is replaced with the corresponding amino acids of the Gαi Protein, having the sequence DCGLF (SEQ ID NO:9). This fusion construct can be obtained by PCR using the following primers:

(SEQ ID NO: 10)
5′-gatcAAGCTTCCATGGCGTGCTGCCTGAGCGAGGAG-3′
and
(SEQ ID NO: 11)
5′-gatcGGATCCTTAGAACAGGCCGCAGTCCTTCAGGTTCAGCTGCAGG
ATGGTG-3′

and Plasmid 63313 which contains the mouse Gαq-wild type version with a hemagglutinin tag as template. Nucleotides in lower caps are included as spacers.

TaqPlus Precision DNA polymerase (Stratagene) can be utilized for the amplification by the following cycles, with steps 2 through 4 repeated 35 times: 95° C. for 2 min; 95° C. for 20 sec; 56° C. for 20 sec; 72° C. for 2 min; and 72° C. for 7 min. The PCR product can be cloned into a pCRII-TOPO vector (Invitrogen) and sequenced using the ABI Big Dye Terminator kit (P.E. Biosystems). Inserts from a TOPO clone containing the sequence of the fusion construct can be shuttled into the expression vector pcDNA3.1(+) at the HindIII/BamHI site by a 2 step cloning process. Also see, PCT Application Number PCT/US02/05625 published as WO02068600 on 6 Sep. 2002, the disclosure of which is hereby incorporated by reference in its entirety.

### Example 13

### [³⁵S]GTPγS Assay

A. Membrane Preparation

In some embodiments membranes comprising the Target GPCR of interest for use in the identification of candidate compounds as, e.g., agonists, inverse agonists or antagonists, are prepared as follows:

a. Materials

“Membrane Scrape Buffer” is comprised of 20 mM HEPES and 10 mM EDTA, pH 7.4; “Membrane Wash Buffer” is comprised of 20 mM HEPES and 0.1 mM EDTA, pH 7.4; “Binding Buffer” is comprised of 20 mM HEPES, 100 mM NaCl, and 10 mM MgCl₂, pH 7.4.

b. Procedure

Firstly, the media is aspirated from a confluent monolayer of cells, followed by rinsing with 5 ml cold PBS, followed by aspiration. Thereafter, 5 ml of Membrane Scrape Buffer is added and cells are scraped off the plate and transferred cells into a 50 ml centrifuge tubes. Cells are then centrifuged at 20,000 rpm for 17 minutes at 4° C. Thereafter, the supernatant is aspirated and the pellet is resuspended in 30 ml Membrane Wash Buffer followed by centrifuge at 20,000 rpm for 17 minutes at 4° C. The supernatant is then aspirated from the membrane pellet. The pellet can be frozen at −80° C. for later use or it can be used immediately and resuspended in Binding Buffer. This is then homogenized using a Brinkman Polytron™ homogenizer (15-20 second bursts until the all material is in suspension). This is referred to herein as “Membrane Protein”.

Bradford Protein Assay

Following the homogenization, protein concentration of the membranes is determined using the Bradford Protein Assay (protein can be diluted to about 1.5 mg/ml, aliquoted and frozen (−80° C.) for later use; when frozen, protocol for use will be as follows: on the day of the assay, frozen Membrane Protein is thawed at room temperature, followed by vortex and then homogenized with a Polytron at about 12×1,000 rpm for about 5-10 seconds; it is noted that for multiple preparations, the homogenizer should be thoroughly cleaned between homogenization of different preparations).

a. Materials

Binding Buffer (as per above); Bradford Dye Reagent; Bradford Protein Standard is utilized, following manufacturer instructions (Biorad, cat. no. 500-0006).

b. Procedure

Duplicate tubes are prepared, one including the membrane, and one as a control “blank”. Each tube contains 800 μl Binding Buffer. Thereafter, 10 μl of Bradford Protein Standard (1 mg/ml) is added to each tube, and 10 μl of membrane Protein is then added to just one tube (not the blank). Thereafter, 200 μl of Bradford Dye Reagent is added to each tube, followed by vortexing of each tube. After five (5) minutes, the tubes are re-vortexed and the material therein is transferred to cuvettes. The cuvettes are read using a CECIL 3041 spectrophotometer, at wavelength 595.

Identification Assay

a. Materials

GDP Buffer consists of 37.5 ml Binding Buffer and 2 mg GDP (Sigma, cat. no. G-7127), followed by a series of dilutions in Binding Buffer to obtain 0.2 μM GDP (final concentration of GDP in each well is 0.1 μM GDP); each well comprising a candidate compound has a final volume of 200 μl consisting of 100 μl GDP Buffer (final concentration, 0.1 μM GDP), 50 μl Membrane Protein in Binding Buffer, and 50 μl [³⁵S]GTPγS (0.6 nM) in Binding Buffer (2.5 μl [³⁵S]GTPγS per 10 ml Binding Buffer).

b. Procedure

Candidate compounds can be screened using a 96-well plate format (these can be frozen at −80° C.). Membrane Protein (or membranes with expression vector excluding the Target GPCR, as control), are homogenized briefly until in suspension. Protein concentration is determined using the Bradford Protein Assay set forth above. Membrane Protein (and control) is diluted to 0.25 mg/ml in Binding Buffer (final assay concentration, 12.5 μg/well).

The membrane protein is diluted to a protein concentration of 0.20 mg/ml in Binding Buffer (20 mM HEPES, pH 7.4, 100 mM NaCl, 10 mM MgCl₂) for a final assay concentration of 5 μg/well. Compound plates to be screened are thawed (daughter plates with 54 compound @ 2 mM in 100% DMSO). The 2 mM compounds are diluted 1:50 with 245 μL GDP buffer (binding buffer plus GDP, Sigma-Aldrich Catalog #87127, ranging from 0.4 to 40 μM, made fresh before assay) to 40 μM in 2% DMSO. Compounds in GDP buffer (25 μl) are added per well of Scintistrip plate (Wallac Catalog #1450-501) and then 50 μl of membrane preparation (0.4 mg protein/ml) is added to each well. The plates are then covered with foil and incubated for 5-10 minutes at room temperature. Subsequently, 25 μl of diluted [³⁵S]GTPγS (Amersham Biosciences Catalog #SJ1320, ˜2000 Ci/mmol—this is made by adding 5 μl [³⁵S]GTPγS stock into 10 ml binding buffer) is added to the wells. The plates are covered with foil and incubated on the shaker (Lab-Line model #1314, at setting of 4) for 60 minutes at room temperature. The assay is stopped by sealing the plates with plate covers and spinning the plates at 4000 rpm for 15 minutes at 22° C. The supernatant of each well is aspirated using an 8-channel manifold and the plate is read in a Wallac Microbeta counter 1450 set up to detect ³⁵S.

### Example 14

### Cyclic AMP Assay

Another assay approach for identifying candidate compounds as, e.g., agonists, inverse agonist, or antagonists, can accomplished by utilizing a cyclase-based assay. In addition to direct identification, this assay approach can be utilized as an independent approach to provide confirmation of the results from the [³⁵S]GTPγS approach as set forth in the above example.

A Homogeneous Time-Resolved Fluorescence (HTRF) Assay for direct cAMP measurement can be utilized for direct identification of candidate compounds as inverse agonists and agonists to a receptor of interest in accordance with the following protocol.

Compounds are screened using HTRF assay for direct cAMP measurement (Gabriel et al, ASSAY and Drug Development Technologies, 1:291-303, 2003) and recombinant cells stably transfected with the receptor of interes. An agonist for a receptor of interest such as the 5-HT₆ receptor is detected in HTRF assay for direct cAMP measurement as a compound which decreases cAMP concentration. HTRF assay also is used to determine EC₅₀ values for 5-HT₆ receptor agonists.

Principle of the assay: HTRF assay kit can be purchased from Cisbio-US, Inc. (Bedford, Mass.; Catalog #62AM4PEC). The HTRF assay supported by the kit is a competitive immunoassay between endogenous cAMP produced by the receptor bearing cells and tracer cAMP labeled with the dye d2. The tracer binding is visualized by a monoclonal anti-cAMP antibody labeled with Cryptate. The specific signal (i.e., fluorescence resonance energy transfer, FRET) is inversely proportional to the concentration of unlabeled cAMP in the standard or sample.

Standard curve: The fluorescence ratio (665 nm/620 nm) of the standards (0.17 to 712 nM cAMP) included in the assay is calculated and used to generate a cAMP standard curve according to the kit manufacturer's instructions. The fluorescence ratio of the samples (test compound or compound buffer) is calculated and used to deduce respective cAMP concentrations by reference to the cAMP standard curve.

Setup of the assay: HTRF assay is carried out using a two-step protocol essentially according to the kit manufacturer's instructions, in 20 μl total volume per well in 384-well plate format (ProxiPlates; PerkinElmer, Fremont, Calif.; catalog #6008280). To each of the experimental wells is transferred 1000 recombinant CHO-K1 cells in 5 μl phosphate buffered saline containing calcium chloride and magnesium chloride (“PBS+”; Invitrogen, Carlsbad, Calif.; catalog #14040) supplemented with IBMX (100 μM) (phosphodiesterase inhibitors; Sigma-Aldrich, St. Louis, Mo.; catalog #I-5879), followed by test compound in 5 μl compound buffer (PBS+ supplemented with 10 μM forskolin (Sigma-Aldrich, St. Louis, Mo.; catalog #F-6886)). The plate is then incubated at room temperature for 1 hour. To each well is then added 5 μl cAMP-d2 conjugate in lysis buffer and 5 μl Cryptate conjugate in lysis buffer according to the kit manufacturer's instructions. The plate is then further incubated at room temperature for 1 hour, after which the assay plate is read.

Assay readout: HTRF® readout is accomplished using a PHERAstar (BMG LABTECH Inc., Durham, N.C.) or EnVision™ (PerkinElmer, Fremont Calif.) microplate reader.

### Example 15

### Fluorometric Imaging Plate Reader (FLIPR) Assay for the Measurement of Intracellular Calcium Concentration

Target Receptor (experimental) and pCMV (negative control) stably transfected cells from respective clonal lines are seeded into poly-D-lysine pretreated 96-well plates (Becton-Dickinson, #356640) at 5.5×10⁴ cells/well with complete culture medium (DMEM with 10% FBS, 2 mM L-glutamine, 1 mM sodium pyruvate) for assay the next day. To prepare Fluo4-AM (Molecular Probe, #F14202) incubation buffer stock, 1 mg Fluo4-AM is dissolved in 467 μl DMSO and 467 μl Pluoronic acid (Molecular Probe, #P3000) to give a 1 mM stock solution that can be stored at −20° C. for a month. Fluo4-AM is a fluorescent calcium indicator dye.

Candidate compounds are prepared in wash buffer (1×HBSS/2.5 mM Probenicid/20 mM HEPES at pH 7.4).

At the time of assay, culture medium is removed from the wells and the cells are loaded with 100 μl of 40 μM Fluo4-AM/2.5 mM Probenicid (Sigma, #P8761)/20 mM HEPES/complete medium at pH 7.4. Incubation at 37° C./5% CO₂ is allowed to proceed for 60 minutes.

After the 1 hour incubation, the Fluo4-AM incubation buffer is removed and the cells are washed 2× with 100 μl wash buffer. In each well is left 100 μl wash buffer. The plate is returned to the incubator at 37° C./5% CO₂ for 60 minutes.

FLIPR (Fluorometric Imaging Plate Reader; Molecular Device) is programmed to add 50 μl candidate compound on the 30th second and to record transient changes in intracellular calcium concentration ([Ca2+]) evoked by the candidate compound for another 150 seconds. Total fluorescence change counts are used to determine agonist activity using the FLIPR software. The instrument software normalizes the fluorescent reading to give equivalent initial readings at zero.

Although the foregoing provides a FLIPR assay for agonist activity using stably transfected cells, a person of ordinary skill in the art would readily be able to modify the assay in order to characterize antagonist activity. Said person of ordinary skill in the art would also readily appreciate that, alternatively, transiently transfected cells could be used.

### Example 16

### MAP Kinase Assay

MAP kinase (mitogen activated kinase) can be monitored to evaluate receptor activation. MAP kinase can be detected by several approaches. One approach is based on an evaluation of the phosphorylation state, either unphosphorylated (inactive) or phosphorylated (active). The phosphorylated protein has a slower mobility in SDS-PAGE and can therefore be compared with the unstimulated protein using Western blotting. Alternatively, antibodies specific for the phosphorylated protein are available (New England Biolabs) which can be used to detect an increase in the phosphorylated kinase. In either method, cells are stimulated with the candidate compound and then extracted with Laemmli buffer. The soluble fraction is applied to an SDS-PAGE gel and proteins are transferred electrophoretically to nitrocellulose or Immobilin. Immunoreactive bands are detected by standard Western blotting technique. Visible or chemiluminescent signals are recorded on film and can be quantified by densitometry.

Another approach is based on evaluation of the MAP kinase activity via a phosphorylation assay. Cells are stimulated with the candidate compound and a soluble extract is prepared. The extract is incubated at 30° C. for 10 minutes with gamma-³²P-ATP, an ATP regenerating system, and a specific substrate for MAP kinase such as phosphorylated heat and acid stable protein regulated by insulin, or PHAS-I. The reaction is terminated by the addition of H₃PO₄ and samples are transferred to ice. An aliquot is spotted onto Whatman P81 chromatography paper, which retains the phosphorylated protein. The chromatography paper is washed and counted for ³²P is a liquid scintillation counter. Alternatively, the cell extract is incubated with gamma-³²P-ATP, an ATP regenerating system, and biotinylated myelin basic protein bound by streptavidin to a filter support. The myelin basic protein is a substrate for activated MAP kinase. The phosphorylation reaction is carried out for 10 minutes at 30° C. The extract can then be aspirated through the filter, which retains, the phosphorylated myelin basic protein. The filter is washed and counted for ³²P by liquid scintillation counting.

### Example 17

### Receptor Binding Assay

In addition to the methods described herein, another means for evaluating a candidate compound is by determining binding affinities to the 5-HT₆ receptor. This type of assay generally requires a radiolabelled ligand to the 5-HT₆ receptor. In addition to the use of known ligands for the 5-HT₆ receptor and radiolabels thereof, 5-HT₆ agonist compounds disclosed herein can be labelled with a radioisotope and used in an assay for evaluating the affinity of a candidate compound to the 5-HT₆ receptor.

A radiolabelled 5-HT₆ compound such as a 5-HT₆ agonist disclosed herein can be used in a screening assay to identify/evaluate compounds. In general terms, a newly synthesized or identified compound (i.e., candidate compound) can be evaluated for its ability to reduce binding of the radiolabelled 5-HT₆ agonist to the 5-HT₆ receptor. Accordingly, the ability to compete with the radiolabelled 5-HT₆ agonist for the binding to the 5-HT₆ receptor directly correlates to the binding affinity of the candidate compound to the 5-HT₆ receptor.

### Assay Protocol for Determining Receptor Binding for 5HT-6:

A. 5-HT₆ Receptor Preparation

For example, HEK293 cells (human kidney, ATCC) can be transiently or stably transfected with 5-HT₆ as described herein. For example, 293 cells can be transiently transfected with 10 μg human 5-HT₆ receptor and 60 μl Lipofectamine (per 15-cm dish), and grown in the dish for 24 hours (75% confluency) with a media change. Cells are removed with 10 ml/dish of Hepes-EDTA buffer (20 mM Hepes +10 mM EDTA, pH 7.4). The cells are then centrifuged in a Beckman Coulter centrifuge for 20 minutes, 17,000 rpm (JA-25.50 rotor). Subsequently, the pellet is resuspended in 20 mM Hepes+1 mM EDTA, pH 7.4 and homogenized with a 50-ml Dounce homogenizer and again centrifuged. After removing the supernatant, the pellets are stored at −80° C., until used in binding assay. When used in the assay, membranes are thawed on ice for 20 minutes and then 10 mL of incubation buffer (20 mM Hepes, 1 mM MgCl₂,100 mM NaCl, pH 7.4) is added. The membranes are then vortexed to resuspend the crude membrane pellet and homogenized with a Brinkmann PT-3100 Polytron homogenizer for 15 seconds at setting 6. The concentration of membrane protein is determined using the BRL Bradford protein assay.

B. Binding Assay

For total binding, a total volume of 50 μl of appropriately diluted membranes (diluted in assay buffer containing 50 mM Tris HCl (pH 7.4), 10 mM MgCl₂, and 1 mM EDTA; 5-50 μg protein) is added to 96-well polyproylene microtiter plates followed by addition of 100 μl of assay buffer and 50 μl of radiolabelled 5-HT₆ agonist. For nonspecific binding, 50 μl of assay buffer is added instead of 100 μl and an additional 50 μl of 10 μM cold 5-HT₆ is added before 50 μl of radiolabelled 5-HT₆ agonist is added. Plates are then incubated at room temperature for 60-120 minutes. The binding reaction is terminated by filtering assay plates through a Microplate Devices GF/C Unifilter filtration plate with a Brandell 96-well plate harvester followed by washing with cold 50 mM Tris HCl, pH 7.4 containing 0.9% NaCl. Then, the bottom of the filtration plates are sealed, 50 μl of Optiphase Supermix is added to each well, the top of the plates are sealed, and plates are counted in a Trilux MicroBeta scintillation counter. For compound competition studies, instead of adding 100 μl of assay buffer, 100 μl of appropriately diluted candidate compound is added to appropriate wells followed by addition of 50 μl of radiolabelled 5-HT₆ agonist.

C. Calculations

The candidate compounds are initially assayed at 1 and 0.1 μM and then at a range of concentrations chosen such that the middle dose would cause about 50% inhibition of a radiolabelled 5-HT₆ agonist binding (i.e., IC₅₀). Specific binding in the absence of candidate compound (B_{O}) is the difference of total binding (B_{T}) minus non-specific binding (NSB) and similarly specific binding (in the presence of candidate compound) (B) is the difference of displacement binding (B_{D}) minus non-specific binding (NSB). IC₅₀ is determined from an inhibition response curve, logit-log plot of % B/B_{O} vs concentration of candidate compound.

Kᵢ is calculated by the Cheng and Prustoff transformation:

Kᵢ=IC₅₀/(1+[L]/K_{D})

where [L] is the concentration of a radiolabelled 5-HT₆ agonist used in the assay and K_{D} is the dissociation constant of a radiolabelled 5-HT₆ agonist determined independently under the same binding conditions.

### Example 18

### Rodent Diabetes Model

Rodent models of type 2 diabetes associated with obesity and insulin resistance have been developed. Genetic models such as db/db and ob/ob [see Diabetes (1982) 31:1-6] in mice and fa/fa in zucker rats have been developed for understanding the pathophysiology of disease and for testing candidate therapeutic compounds [Diabetes (1983) 32:830-838; Annu Rep Sankyo Res Lab (1994) 46:1-57]. The homozygous animals, C57 BL/KsJ-db/db mice developed by Jackson Laboratory are obese, hyperglycemic, hyperinsulinemic and insulin resistant [J Clin Invest (1990) 85:962-967], whereas heterozygotes are lean and normoglycemic. In the db/db model, mice progressively develop insulinopenia with age, a feature commonly observed in late stages of human type 2 diabetes when sugar levels are insufficiently controlled. Since this model resembles that of human type 2 diabetes, the compounds of the present invention are tested for activities including, but not limited to, lowering of plasma glucose and triglycerides. Zucker (fa/fa) rats are severely obese, hyperinsulinemic, and insulin resistant {Coleman, Diabetes (1982) 31:1; E Shafrir in Diabetes Mellitus, H Rifkin and D Porte, Jr, Eds [Elsevier Science Publishing Co, New York, ed. 4, (1990), pp. 299-340]}, and the fa/fa mutation may be the rat equivalent of the murine db mutation [Friedman et al, Cell (1992) 69:217-220; Truett et al, Proc Natl Acad Sci USA (1991) 88:7806]. Tubby (tub/tub) mice are characterized by obesity, moderate insulin resistance and hyperinsulinemia without significant hyperglycemia [Coleman et al, Heredity (1990) 81:424].

The present invention encompasses the use 5-HT₆ receptor modulators for reducing the insulin resistance and hyperglycemia in any or all of the above rodent diabetes models, in humans with type 2 diabetes or other preferred insulin-related disorders or disorders of lipid metabolism described previously, or in models based on other mammals. Plasma glucose and insulin levels can be tested, as well as other factors including, but not limited to, plasma free fatty acids and triglycerides. In Vivo Assay for Anti-Hyperglycemic Activity of 5-HT₆ receptor modulators

Genetically altered obese diabetic mice (db/db) (male, 7-9 weeks old) are housed (7-9 mice/cage) under standard laboratory conditions at 22° C. and 50% relative humidity, and maintained on a diet of Purina rodent chow and water ad libitum. Prior to treatment, blood is collected from the tail vein of each animal and blood glucose concentrations are determined using One Touch Basic Glucose Monitor System (Lifescan). Mice that have plasma glucose levels between 250 to 500 mg/dl are used. Each treatment group consists of seven mice that are distributed so that the mean glucose levels are equivalent in each group at the start of the study. db/db mice are dosed by micro-osmotic pumps, inserted using isoflurane anesthesia, to provide compounds of the invention, saline, or an irrelevant compound to the mice subcutaneously (s.c.). Blood is sampled from the tail vein at intervals thereafter and analyzed for blood glucose concentrations. Significant differences between groups (comparing compounds of the interest to saline-treated) are evaluated using Student t-test.

The foregoing is provided by way of illustration and not limitation. Other illustrative rodent models for type 2 diabetes have been described [Moller D E, Nature (2001) 414:821-7 and references therein; and Reed M J et al., Diabetes, Obesity and Metabolism (1999) 1:75-86 and reference therein; the disclosure of each of which is hereby incorporated by reference in its entirety].

### Example 19

### Mouse Atherosclerosis Model

Adiponectin-deficient mice generated through knocking out the adiponectin gene have been shown to be predisposed to atherosclerosis and to be insulin resistant. The mice are also a suitable model for ischemic heart disease [Matsuda, M et al. J Biol Chem (2002) July, and references cited therein, the disclosures of which are incorporated herein by reference in their entirety].

Adiponectin knockout mice are housed (7-9 mice/cage) under standard laboratory conditions at 22° C. and 50% relative humidity. The mice are dosed by micro-osmotic pumps, inserted using isoflurane anesthesia, to provide compounds of the invention, saline, or an irrelevant compound to the mice subcutaneously (s.c.). Neointimal thickening and ischemic heart disease are determined for different groups of mice sacrificed at different time intervals. Significant differences between groups (comparing compounds of the interest to saline-treated) are evaluated using Student t-test.

The foregoing mouse model of atherosclerosis is provided by way of illustration and not limitation. By way of further example, Apolipoprotein E-deficient mice have also been shown to be predisposed to atherosclerosis [Plump A S et al., Cell (1992) 71:343-353; the disclosure of which is hereby incorporated by reference in its entirety].

Another model that can be used is that of diet-induced atherosclerosis in C57BL/6J mice, an inbred strain known to be susceptible to diet-induced atherosclerotic lesion formation. This model is well known to persons of ordinary skill in the art [Kamada N et al., J Atheroscler Thromb (2001) 8:1-6; Garber D W et al., J Lipid Res (2001) 42:545-52; Smith J D et al., J Intern Med (1997) 242:99-109; the disclosure of each of which is hereby incorporated by reference in its entirety].

### Example 20

### In Vivo Pig Model of HDL-Cholesterol and Atherosclerosis

The utility of a compound of interest as a medical agent in the prevention or treatment of a high total cholesterol/HDL-cholesterol ratio and conditions relating thereto is demonstrated, for example, by the activity of the compound in lowering the ratio of total cholesterol to HDL-cholesterol, in elevating HDL-cholesterol, or in protection from atherosclerosis in an in vivo pig model. Pigs are used as an animal model because they reflect human physiology, especially lipid metabolism, more closely than most other animal models. An illustrative in vivo pig model not intended to be limiting is presented here.

Yorkshire albino pigs (body weight 25.5±4 kg) are fed a saturated fatty acid rich and cholesterol rich (SFA-CHO) diet during 50 days (1 kg chow 35 kg-1 pig weight), composed of standard chow supplemented with 2% cholesterol and 20% beef tallow [Royo T et al., European Journal of Clinical Investigation (2000) 30:843-52]. Saturated to unsaturated fatty acid ratio is modified from 0.6 in normal pig chow to 1.12 in the SFA-CHO diet. Animals are divided into two groups, one group (n=8) fed with the SFA-CHO diet and treated with placebo and one group (n=8) fed with the SFA-CHO diet and treated with the modulator (3.0 mg kg-1). Control animals are fed a standard chow for a period of 50 days. Blood samples are collected at baseline (2 days after the reception of the animals), and 50 days after the initiation of the diet. Blood lipids are analyzed. The animals are sacrificed and necropsied.

Alternatively, the foregoing analysis comprises a plurality of groups each treated with a different dose of the compound of interest. Doses include, for example: 0.1 mg kg-1, 0.3 mg kg-1, 1.0 mg kg-1, 3.0 mg kg-1, 10 mg kg-1, 30 mg kg-1 and 100 mg kg-1. Alternatively, the foregoing analysis is carried out at a plurality of timepoints, for example, 10 weeks, 20 weeks, 30 weeks, 40 weeks, and 50 weeks.

### HDL-Cholesterol

Blood is collected in trisodium citrate (3.8%, 1:10). Plasma is obtained after centrifugation (1200 g 15 min) and immediately processed. Total cholesterol, HDL-cholesterol, and LDL-cholesterol are measured using the automatic analyzer Kodak Ektachem DT System (Eastman Kodak Company, Rochester, N.Y., USA). Samples with value parameters above the range are diluted with the solution supplied by the manufacturer and then re-analyzed. The total cholesterol/HDL-cholesterol ratio is determined. Comparison is made of the level of HDL-cholesterol between groups. Comparison is made of the total cholesterol/HDL-cholesterol ratio between groups.

Elevation of HDL-cholesterol or reduction of the total cholesterol/HDL-cholesterol ratio on administration of the compound of interest is taken as indicative of the compound having the aforesaid utility.

### Atherosclerosis

The thoracic and abdominal aortas are removed intact, opened longitudinally along the ventral surface, and fixed in neutral-buffered formalin after excision of samples from standard sites in the thoracic and abdominal aorta for histological examination and lipid composition and synthesis studies. After fixation, the whole aortas are stained with Sudan IV and pinned out flat, and digital images are obtained with a TV camera connected to a computerized image analysis system (Image Pro Plus; Media Cybernetics, Silver Spring, Md.) to determine the percentage of aortic surface involved with atherosclerotic lesions [Gerrity R G et al, Diabetes (2001) 50:1654-65; Cornhill J F et al, Arteriosclerosis, Thrombosis, and Vascular Biology (1985) 5:415-26; which disclosures are hereby incorporated by reference in their entirety]. Comparison is made between groups of the percentage of aortic surface involved with atherosclerotic lesions.

Reduction of the percentage of aortic surface involved with atherosclerotic lesions on administration of the compound of interest is taken as indicative of the compound having the aforesaid utility.

### Plasma Free Fatty Acids

It would be readily apparent to one of ordinary skill in the art that the foregoing in vivo pig model is easily modified in order to address, without limitation, the activity of a compound in lowering plasma free fatty acids.

Those skilled in the art will recognize that various modifications, additions, substitutions, and variations to the illustrative examples set forth herein can be made without departing from the spirit of the invention and are, therefore, considered within the scope of the invention. All documents referenced above, including, but not limited to, printed publications, and provisional and regular patent applications, are incorporated herein by reference in their entirety.

1. A method for identifying a glycemic stabilizing compound, comprising:
a) contacting a candidate compound with a 5-HT6 receptor,
b) determining whether 5-HT6 receptor functionality is increased, and
c) determining whether a glycemic marker is directly modulated, wherein an increase in 5-HT6 receptor functionality and direct modulation of a glycemic marker identifies the candidate compound as said glycemic stabilizing compound.

2. The method of claim 1, wherein the modulation of the glycemic marker is first detected within less than 24 hours of the contacting of the candidate compound with the 5-HT6 receptor.

3. A method for identifying a glycemic stabilizing compound, comprising:
a) contacting a single dose of a candidate compound with a 5-HT6 receptor,
b) determining whether 5-HT6 receptor functionality is increased, and
c) determining whether a glycemic marker is directly modulated, wherein an increase in 5-HT6 receptor functionality and direct modulation of a glycemic marker identifies the candidate compound as said glycemic stabilizing compound.

4. A method for identifying a glycemic stabilizing compound, comprising:
a) contacting a candidate compound with a 5-HT6 receptor in an isolated cell,
b) determining whether 5-HT6 receptor functionality is increased, and
c) determining whether a glycemic marker is directly modulated, wherein an increase in 5-HT6 receptor functionality and direct modulation of a glycemic marker identifies the candidate compound as said glycemic stabilizing compound.

5. The method of claim 1, wherein said 5-HT6 receptor is human.

6. The method of claim 1, wherein said determining comprises a second messenger assay.

7. The method of claim 1, wherein said glycemic stabilizing compound decreases blood glucose concentration.

8. The method of claim 1, wherein said glycemic stabilizing compound increases insulin secretion.

9. The method of claim 1, wherein said glycemic marker is glucose.

10. The method of claim 1, wherein said glycemic marker is insulin.

11. A method for treating or preventing an insulin-related disorder in an individual in need thereof, comprising administering to said individual an effective amount of the glycemic stabilizing compound of claim 1.

12. The method of claim 11, wherein said insulin-related disorder is insulin resistance, impaired glucose tolerance, or diabetes.

13. The method of claim 11, further comprising administering to said individual an effective amount of an agent used for the treatment of diabetes, blood lipid disorders, or obesity in combination with an effective amount of the glycemic stabilizing compound of claim 1.

14. The method of claim 11, wherein the individual is a human.

15. A method for decreasing blood glucose levels in an individual in need thereof, comprising administering to the individual an effective amount of the glycemic stabilizing compound of claim 1.

16. A method for increasing insulin secretion in an individual in need thereof, comprising administering to the individual an effective amount of the glycemic stabilizing compound of claim 1.

17. The method of claim 16, wherein said increasing insulin secretion in an individual is in a glucose dependent manner.
